(19) United States
(12) Patent Application Publication
(10) Pub. No.: US 2018/0353398 A1
(43) Pub. Date: Dec. 13, 2018

(54) HAIR CARE REGIMEN USING NA AEROSOL FOAM CONCENTRATED CONDITIONER

(71) Applicant: The Procter & Gamble Company, Cincinnati, OH (US)

(72) Inventors: Jazmin Veronica Torres Rivera, Liberty Township, OH (US); Kathleen Mary Kaufman, Cincinnati, OH (US); Robert Wayne Glenn, JR., Liberty Township, OH (US)

(21) Appl. No.: 16/104,343

(22) Filed: Aug. 17, 2018

Related U.S. Application Data
(63) Continuation of application No. 15/135,684, filed on Apr. 22, 2016.
(60) Provisional application No. 62/151,645, filed on Apr. 23, 2015.

(51) Int. Cl. A61K 8/06; A61K 8/20; A61K 8/898; A61Q 5/12; A61K 8/60; A61Q 5/02; A61K 8/46; A61K 8/44; A61K 8/41; A61K 8/34; A61K 8/35; A61K 8/365; A61K 8/58; A61K 8/73; A61K 8/81; A61K 8/04

(52) U.S. Cl. CPC A61K 8/068; A61K 2800/413; A61K 8/046; A61K 8/898; A61Q 5/12; A61K 8/604; A61Q 5/02; A61K 8/463; A61K 8/44; A61K 8/416; A61K 8/342; A61K 8/35; A61K 8/365; A61K 8/442; A61K 2800/87; A61K 8/602; A61K 8/585; A61K 8/737; A61K 8/8129; A61K 8/817; A61K 2800/21; A61K 2800/30; A61K 2800/596; A61K 2800/80; A61K 8/20

(57) ABSTRACT

A method of treating the hair including applying to the hair a shampoo composition, rinsing the shampoo composition from the hair, applying to the hair a concentrated conditioner composition, and rinsing the concentrated conditioner composition from the hair. The shampoo composition includes an anionic surfactant, one or more amphoteric or zwitterionic co-surfactants, and less than 0.25% of one or more shampoo high melting point fatty compounds. The concentrated conditioner composition includes one or more silicones, perfume, and less than 10% high melting point fatty compounds.

### FIELD OF THE INVENTION

Described herein is a method of treating hair with a shampoo composition and an aerosol foam concentrated hair conditioning composition having less than 5% of one or more conditioner high melting point fatty compounds, by weight of the concentrated conditioner composition.

### BACKGROUND OF THE INVENTION

Today's hair conditioners almost universally comprise high levels of high melting point fatty compounds, the most common of which are C16 to C18 fatty alcohols. These high melting point fatty compounds are employed as structuring agents wherein they are combined with one or more surfactants and an aqueous carrier to form a gel network. The gel network provides a viscous and high yield point rheology which facilitates the dispensing of the conditioner from a bottle or tube and the subsequent distribution and spreading of the product through the hair by the consumer. The gel network structuring also enables incorporation of silicones, perfumes and oils in the form of an oil-in-water emulsion that is phase stable. These silicones and oils are intended to be deposited on the hair to provide the primary hair conditioning benefits including wet and dry combing friction reduction and hair manageability etc.

However, today's gel network hair conditioners lead to excessive co-deposits of the high melting point fatty compound on the hair over multiple cycles. Additionally, the deposited high melting point fatty compounds build-up on hair over multiple cycles and lead to significant waxy build-up on hair and hair weigh down. Indeed, one of the major consumer complaints with hair conditioners is waxy residue which makes hair look greasy or feel heavy. Many current gel network hair conditioners deposit significantly more high melting point fatty compounds (fatty alcohols) than silicone or oil after multiple treatment cycles in technical testing. While not being bound to theory, this is hypothesized to be due to the higher concentration of high melting point weight fatty compounds in the product relative to the silicone or oil. Importantly, such a high level of melting point fatty compounds (fatty alcohols) is required to produce a shelf stable gel network with sufficient structuring for consumer acceptable viscosity and rheology.

Described herein is a concentrated hair care composition that enables new product opportunities and consumer benefits by addressing the current disadvantages associated with gel network conditioners. Is has been found that concentrated and ultra-low viscosity hair conditioner compositions can be delivered to the hair in foamed form. These new concentrated silicone nanoemulsion compositions enable sufficient dosage from a foam delivery form while also eliminating the need for high melting point fatty compounds or other “insoluble” structurants that can lead to significant co-deposits, build-up and weigh down of hair. The net result has been a step change improvement in silicone deposition purity versus today's rinse-off products and an improvement in technical performance benefits from such a pure and transparent deposited silicone layer. These benefits include multicycle hair conditioning without hair weigh down, durable conditioning, reduced hair dye fade, and increased color vibrancy.

Nanoemulsion technology development is hindered by complex stability issues that emerge when droplet sizes are driven to the nanoscale. This is especially problematic in the presence of higher levels of perfume oils required for such a concentrated product. The concentrated hair care composition described herein is therefor also focused on improved stability.

### SUMMARY OF THE INVENTION

Described herein is a method of treating the hair, the method comprising (a) applying to the hair a shampoo composition comprising (i) from about 8% to about 40% of one or more anionic surfactants, by weight of the shampoo composition; and (ii) from about 0.5% to about 15% of a co-surfactant selected from the group consisting of amphoteric, non-ionic, zwitterionic, and combinations thereof; wherein the shampoo composition comprises less than 0.25% of one or more shampoo high melting point fatty compounds; (b) rinsing the shampoo composition from the hair; (c) applying to the hair a concentrated conditioner composition dispensed from an aerosol foam dispenser as a dosage of foam, wherein the concentrated conditioner composition comprises (i) from about 4% to about 22% of one or more oils, by weight of the concentrated conditioner composition, wherein the particle size of the one or more oils is from about 1 nm to about 300 nm; (ii) less than 4% of one or more conditioner high melting point fatty compounds, by weight of the concentrated conditioner composition; (iii) from about 1% to about 10% propellant, by weight of the concentrated conditioner composition; (iv) from about 0.5% to about 7% perfume, by weight of the concentrated conditioner composition; and (v) from about 50% to about 95% water, by weight of the concentrated conditioner composition; wherein the concentrated conditioner composition has a liquid phase viscosity of from about 1 centipoise to about 3,000 centipoise; wherein the concentrated conditioner composition has a silicone to conditioner high melting point fatty compound weight ratio of from about 100:0 to about 50:50; wherein the concentrated conditioner composition has a silicone to perfume weight ratio of from about 98:2 to about 50:50; and wherein the foam has a density of from about 0.025 g/cm³ to about 0.30 g/cm³ when dispensed from the foam dispenser; and (d) rinsing the concentrated conditioner composition from the hair; wherein the method has a deposition purity of from about 40% to about 100%.

### DETAILED DESCRIPTION OF THE INVENTION

While the specification concludes with claims particularly pointing out and distinctly claiming the invention, it is believed that the present invention will be better understood from the following description.

As used herein, the articles including “a” and “an” when used in a claim, are understood to mean one or more of what is claimed or described.

As used herein, “comprising” means that other steps and other ingredients which do not affect the end result can be added. This term encompasses the terms “consisting of” and “consisting essentially of”.

As used herein, “mixtures” is meant to include a simple combination of materials and any compounds that may result from their combination.

As used herein, “molecular weight” or “M.Wt.” refers to the weight average molecular weight unless otherwise stated.

As used herein, the terms “include,” “includes,” and “including,” are meant to be non-limiting and are understood to mean “comprise,” “comprises,” and “comprising,” respectively.

As used herein, the term “concentrated” means a conditioner composition comprising from about 4% to about 22% of one or more oils, by weight of the concentrated conditioner composition.

As used herein, the term “nanoemulsion” means an oil-in-water (o/w) emulsion with an average particle size ranging from about 1 nm to about 100 nm. The particle size referred to herein is z-average measured by dynamic light scattering. The nanoemulsion described herein may be prepared by the following methods: (1) mechanically breaking down the emulsion droplet size; (2) spontaneously forming the emulsion (may be referred to as a microemulsion in the literature); and (3) using emulsion polymerization to achieve average particle size in the target range described herein.

As used herein, the term “viscosity reducing agent” means organic compounds having a molecular weight of from about 100 to about 300 daltons, alternatively from about 125 daltons to about 300 daltons. Additionally, the viscosity reducing agents may have a water solubility at between 23 and 25 degrees Celsius of from about 900 to 50,000 mg/L.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Unless otherwise noted, all component or composition levels are in reference to the active portion of that component or composition, and are exclusive of impurities, for example, residual solvents or by-products, which may be present in commercially available sources of such components or compositions.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Method of Treating Hair

The method of treating the hair described herein comprises applying to the hair a shampoo composition, rinsing the shampoo composition from the hair, applying to the hair a concentrated conditioner composition, and rinsing the concentrated conditioner composition from the hair. The shampoo composition may include one or more anionic surfactants, one or more amphoteric, non-ionic, or zwitterionic co-surfactants, and less than 0.25% of one or more shampoo high melting point fatty compounds. The concentrated conditioner composition may include one or more silicones, perfume, and less than 5% high melting point fatty compounds.

### Shampoo Composition

A. Surfactants

The shampoo composition may comprise from about 8% to about 40%, alternatively from about 16% to about 40%, alternatively from about 18% to about 36%, alternatively from about 20% to about 32%, alternatively from about 22% to about 28% of one or more anionic surfactants, by weight of the shampoo composition. In an embodiment, the shampoo composition may comprise from about 8% to about 20%, alternatively from about 10% to about 18%, alternatively from about 12% to about 16% of one or more anionic surfactants, by weight of the shampoo composition.

Anionic surfactants suitable for use in the compositions are the alkyl and alkyl ether sulfates. Other suitable anionic surfactants are the water-soluble salts of organic, sulfuric acid reaction products. Still other suitable anionic surfactants are the reaction products of fatty acids esterified with isethionic acid and neutralized with sodium hydroxide. Other similar anionic surfactants are described in U.S. Pat. Nos. 2,486,921; 2,486,922; and 2,396,278, which are incorporated herein by reference in their entirety.

Exemplary anionic surfactants for use in the hair care composition include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. In a further embodiment, the anionic surfactant is sodium lauryl sulfate or sodium laureth sulfate.

Suitable anionic surfactants include, but are not limited to undecyl sulfate compound selected from the group consisting of:
a) R₁O(CH₂CHR₃O)_{y}SO₃M;
b) CH₃(CH₂)_{z}CHR₂CH₂O(CH₂CHR₃O)_{y}SO₃M; and
c) mixtures thereof,

where Ri represents CH₃(CH₂)₁₀, R₂ represents H or a hydrocarbon radical comprising 1 to 4 carbon atoms such that the sum of the carbon atoms in z and R₂ is 8, R₃ is H or CH₃, y is 0 to 7, the average value of y is about 1 when y is not zero (0), and M is a monovalent or divalent, positively-charged cation.

Suitable anionic alkyl sulfates and alkyl ether sulfate surfactants include, but are not limited to, those having branched alkyl chains which are synthesized from C8 to C18 branched alcohols which may be selected from the group consisting of: Guerbet alcohols, aldol condensation derived alcohols, oxo alcohols and mixtures thereof. Non-limiting examples of the 2-alkyl branched alcohols include oxo alcohols such as 2-methyl-1-undecanol, 2-ethyl-1-decanol, 2-propyl-1-nonanol, 2-butyl 1-octanol, 2-methyl-1-dodecanol, 2-ethyl-1-undecanol, 2-propyl-1-decanol, 2-butyl-1-nonanol, 2-pentyl-1-octanol, 2-pentyl-1-heptanol, and those sold under the tradenames LIAL® (Sasol), ISALCHEM® (Sasol), and NEODOL® (Shell), and Guerbet and aldol condensation derived alcohols such as 2-ethyl-1-hexanol, 2-propyl-1-butanol, 2-butyl-1-octanol, 2-butyl-1-decanol, 2-pentyl-1-nonanol, 2-hexyl-1-octanol, 2-hexyl-1-decanol and those sold under the tradename ISOFOL® (Sasol) or sold as alcohol ethoxylates and alkoxylates under the tradenames LUTENSOL XP® (BASF) and LUTENSOL XL® (BASF).

The anionic alkyl sulfates and alkyl ether sulfates may also include those synthesized from C8 to C18 branched alcohols derived from butylene or propylene which are sold under the trade names EXXAL™ (Exxon) and Marlipal® (Sasol). This includes anionic surfactants of the subclass of sodium trideceth-n sulfates (STnS), where n is between about 0.5 and about 3.5. Exemplary surfactants of this subclass are sodium trideceth-2 sulfates and sodium trideceth-3 sulfates. The composition of the present invention can also include sodium tridecyl sulfate.

The shampoo composition may comprise from about 0.25% to about 15%, alternatively from about 0.5% to about 14%, alternatively from about 0.5% to about 13%, alternatively from about 1% to about 12%, alternatively from about 0.5% to about 10%, alternatively from about 3% to about 10%, alternatively from about 4% to about 9% of one or more amphoteric, nonionic, or zwitterionic co-surfactants, by weight of the shampoo composition. In an embodiment, the shampoo composition may comprise from about 0.25% to about 10%, alternatively from about 0.5% to about 8%, alternatively from about 0.75% to about 6%, alternatively from about 1% to about 4%, alternatively from about 1.25% to about 2% of one or more amphoteric, nonionic, or zwitterionic co-surfactants, by weight of the shampoo composition. The co-surfactant can include, but is not limited to, lauramidopropyl betaine, cocoamidopropyl betaine, lauryl hydroxysultaine, sodium lauroamphoacetate, coco monoethanolamide and mixtures thereof. The shampoo composition may comprise from about 0.25% to about 15%, alternatively from about 2% to about 14%, alternatively from about 0.5% to about 10%, alternatively from about 3% to about 10%, alternatively from about 4% to about 9% of one or more amphoteric or zwitterionic co-surfactants, by weight of the shampoo composition.

Suitable amphoteric or zwitterionic surfactants for use in the hair care composition described herein include those which are known for use in shampoo or other hair care cleansing. Non limiting examples of suitable zwitterionic or amphoteric surfactants are described in U.S. Pat. Nos. 5,104,646 and 5,106,609, which are incorporated herein by reference in their entirety.

Amphoteric co-surfactants suitable for use in the composition include those surfactants described as derivatives of aliphatic secondary and tertiary' amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Suitable amphoteric surfactant include, but are not limited to, thoseselected from the group consisting of: sodium cocaminopropionate, sodium cocaminodipropionate, sodium cocoamphoacetate,sodium cocoamphohydroxypropylsulfonate, sodium cocoamphopropionate, sodium comamphopropionate, sodium lauraminopropionate, sodium lauroamphoacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphohydroxypropylsulfonate, ammonium cocoamphopropionate, ammonium cornamphopropionate, ammonium lauraminopropionate, ammonium lauroamphoacetate, ammonium lauroamphohydroxypropylsulfonate, ammonium lauroamphopropionate, ammonium cocoamphopropionate, ammonium lauriminodipropionate, triethanonlamine cocaminopropionate, triethanonlamine cocaminodipropionate, triethanoniamine cocoamphoacetate, triethanonlamine cocoamphohydroxypropylsulfonate, triethanonlamine cocoamphopropionate, triethanonlamine cornamphopropionate, triethanonlamine lauraminopropionate, triethanonlamine lauroamphoacetate, triethanonlamine lauroamphohydroxypropylsulfonate, triethanonlamine lauroamphopropionate, trethanonlamine cornamphopropionate, triethanonlamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarbox yethylhydroxypropylsuifonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethyl-7 carboxyamphodiacetate, lauraminopropionic acid, lauroamphodipropionic acid, lauryl aminopropylglycine, lauryl diethylenediaminoglycine, and mixtures thereof.

The amphoteric co-surfactant can be a surfactant according to the following structure:

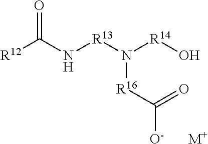

wherein R12 is a C-linked monovalent substituent selected from the group consisting of substituted alkyl systems comprising 9 to 15 carbon atoms, unsubstituted alkyl systems comprising 9 to 15 carbon atoms, straight alkyl systems comprising 9 to 15 carbon atoms, branched alkyl systems comprising 9 to 15 carbon atoms, and unsaturated alkyl systems comprising 9 to 15 carbon atoms; R13, R14, and R15 are each independently selected from the group consisting of C-linked divalent straight alkyl systems comprising 1 to 3 carbon atoms, and C-linked divalent branched alkyl systems comprising 1 to 3 carbon atoms; and M+ is a monovalent counterion selected from the group consisting of sodium, ammonium and protonated triethanolamine. In an embodiment, the amphoteric surfactant is selected from the group consisting of: sodium cocoamphoacetate, sodium cocoamphodiacetate, sodium lauroamphoacetate, sodium lauroamphodiacetate, ammonium lauroamphoacetate, ammonium cocoamphoacetate, triethanolamine lauroamphoacetate, triethanolamine cocoamphoacetate, and mixtures thereof.

The shampoo composition may comprises a zwitterionic co-surfactant, wherein the zwitterionic surfactant is a derivative of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. The zwitterionic surfactant can be selected from the group consisting of: cocamid.oethyl betaine, cocarnidopropylamine oxide, cocamidopropyl betaine, cocamidopropyl dimethylaminohydroxypropyl hydrolyzed collagen, cocarnidopropyldimonium hydroxypropyl hydrolyzed collagen, cocamidopropyl hydroxysultaine, cocobetaineamido amphopropionate, coco-betaine, coco-hydroxysultaine, coco/oleamidopropyl betaine, coco-sultaine, lauramidopropyl betaine, lauryl betaine, lauryl hydroxysultaine, lauryl sultaine, and mixtures thereof. A suitable zwitterionic surfactant is lauryl hydroxysultaine. The zwitterionic surfactant can be selected from the group consisting of: lauryl hydroxysultaine, cocamidopropyl hydroxysultaine, coco-betaine, coco-hydroxysultaine, coco-sultaine, lauryl betaine, lauryl sultaine, and mixtures thereof.

The co-surfactant can be a zwitterionic surfactant, wherein the zwitterionic surfactant is selected from the group consisting of: lauryl hydroxysultaine, cocamidopropyl hydroxysultaine, coco-betaine, coco-hydroxysultaine, coco-sultaine, lauryl betaine, lauryl sultaine, and mixtures thereof.

In a preferred embodiment, the co-surfactant is selected from amphoteric or zwitterionic surfactants synthesized from lauric acid including, but not limited to, lauramidopropyl betaine, lauryl Hydroxysultaine, and sodium lauroamphoacetate and having a chain length distribution wherein the C12 chain length averages from about 80% to about 100%, alternatively from about 85% to about 100%, alternatively from about 90% to about 100%, alternatively from about 95% to about 100%, and alternatively from about 97% to about 100% of the total molecular chain length distribution.

Suitable nonionic surfactants for use in the hair care composition include those described in McCutcheion's Detergents and Emulsifiers, North American edition (1986), Allured Publishing Corp., and McCutcheion's Functional Materials, North American edition (1992). Suitable nonionic surfactants for use in the hair care composition include, but are not limited to, polyoxyethylenated alkyl phenols, polyoxyethylenated alcohols, polyoxyethylenated polyoxypropylene glycols, glyceryl esters of alkanoic acids, polyglyceryl esters of alkanoic acids, propylene glycol esters of alkanoic acids, sorbitol esters of alkanoic acids, polyoxyethylenated sorbitor esters of alkanoic acids, polyoxyethylene glycol esters of alkanoic acids, polyoxyethylenated alkanoic acids, alkanolamides, N-alkylpyrrolidones, alkyl glycosides, alkyl polyglucosides, alkylamine oxides, and polyoxyethylenated silicones.

The non-ionic surfactant may be selected from the group consisting of: Cocamide,Cocamide Methyl MEA, Cocamide DEA, Cocamide MEA, Cocamide MIPA, Lauramide DEA, Lauramide MEA, Lauramide MIPA, Myristamide DEA, Myristamide MEA, PEG-20 Cocamide MEA, PEG-2 Cocamide, PEG-3 Cocamide, PEG-4 Cocamide, PEG-5 Cocamide, PEG-6 Cocamide, PEG-7 Cocamide, PEG-3 Lauramide, PEG-5 Lauramide, PEG-3 Oleamide, PPG-2 Cocamide, PPG-2 Hydroxyethyl Cocamide, and mixtures thereof.

Non limiting examples of other anionic, zwitterionic, non-ionic, and amphoteric additional surfactants suitable for use in the hair care composition are described in McCutcheon's, Emulsifiers and Detergents, 1989 Annual, published by M. C. Publishing Co., and U.S. Pat. Nos. 3,929,678, 2,658,072; 2,438,091; 2,528,378, which are incorporated herein by reference in their entirety.

B. Shampoo High Melting Point Fatty Compounds

The shampoo composition may comprise less than 1%, alternatively less than 0.5%, alternatively less than 0.25% shampoo high melting point fatty compounds, by weight of the shampoo composition. The shampoo composition may be substantially free of shampoo high melting point fatty compounds, and alternatively may comprise 0% shampoo high melting point fatty compounds, by weight of the shampoo composition.

The high melting point fatty compounds have a melting point of about 25° C. or higher, and are selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than about 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

The fatty alcohols described herein are those having from about 14 to about 30 carbon atoms, preferably from about 16 to about 22 carbon atoms. These fatty alcohols are saturated and can be straight or branched chain alcohols. Nonlimiting examples of fatty alcohols include cetyl alcohol, stearyl alcohol, behenyl alcohol, and mixtures thereof.

The fatty acids useful herein are those having from about 10 to about 30 carbon atoms, preferably from about 12 to about 22 carbon atoms, and more preferably from about 16 to about 22 carbon atoms. These fatty acids are saturated and can be straight or branched chain acids. Also included are diacids, triacids, and other multiple acids which meet the requirements herein. Also included herein are salts of these fatty acids. Nonlimiting examples of fatty acids include lauric acid, palmitic acid, stearic acid, behenic acid, sebacic acid, and mixtures thereof.

The fatty alcohol derivatives and fatty acid derivatives useful herein include alkyl ethers of fatty alcohols, alkoxylated fatty alcohols, alkyl ethers of alkoxylated fatty alcohols, esters of fatty alcohols, fatty acid esters of compounds having esterifiable hydroxy groups, hydroxy-substituted fatty acids, and mixtures thereof. Nonlimiting examples of fatty alcohol derivatives and fatty acid derivatives include materials such as methyl stearyl ether; the ceteth series of compounds such as ceteth-1 through ceteth-45, which are ethylene glycol ethers of cetyl alcohol, wherein the numeric designation indicates the number of ethylene glycol moieties present; the steareth series of compounds such as steareth-1 through steareth-10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e., a mixture of fatty alcohols containing predominantly cetyl and stearyl alcohol, wherein the numeric designation indicates the number of ethylene glycol moieties present; C16-C30 alkyl ethers of the ceteth, steareth, and ceteareth compounds just described; polyoxyethylene ethers of behenyl alcohol; ethyl stearate, cetyl stearate, cetyl palmitate, stearyl stearate, myristyl myristate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, glyceryl monostearate, glyceryl distearate, glyceryl tristearate, ethylene glycol distearate; hydroxyl containing derivatives such as 12-hydroxystearic acid, 9,10-dihydroxystearic acid, tri-9,10-dihydroxystearin and tri-12-hydroxystearin (hydrogenated castor oil is mostly tri-12-hydroxystearin) and mixtures thereof.

C. Cationic Polymers

The shampoo composition described herein may also comprise one or more cationic polymers. These cationic polymers may be selected from the group consisting of cationic guar polymers, cationic non-guar galactomannan polymers, cationic tapioca polymers, cationic copolymers of acrylamide monomers and cationic monomers, synthetic non-crosslinked cationic polymers which may or may not form lyotropic liquid crystals upon combination with the detersive surfactant, cationic cellulose polymers, and combinations thereof. The hair care composition may comprise a cationic polymer selected from the group consisting of guar polymers, non-guar galactomannan polymers, tapioca polymers, copolymers of acrylamide monomers and cationic monomers, cellulose polymers, and combinations thereof.

The shampoo composition may comprise a cationic guar polymer, which is a cationically substituted galactomannan (guar) gum derivatives. Guar gum for use in preparing these guar gum derivatives may be obtained as a naturally occurring material from the seeds of the guar plant. The guar molecule itself is a straight chain mannan, which is branched at regular intervals with single membered galactose units on alternative mannose units. The mannose units are linked to each other by means of β(1-4) glycosidic linkages. The galactose branching arises by way of an α(1-6) linkage. Cationic derivatives of the guar gums are obtained by reaction between the hydroxyl groups of the polygalactomannan and reactive quaternary ammonium compounds. The degree of substitution of the cationic groups onto the guar structure should be sufficient to provide the requisite cationic charge density described above.

The cationic polymer, including but not limited to a cationic guar polymer, may have a molecular weight of less than 1.0 million g/mol, or from about 10 thousand to about 1 million g/mol, or from about 25 thousand to about lmillion g/mol, or from about 50 thousand to about 1 million g/mol, or from about 100 thousand to about 1 million g/mol. In one embodiment, the cationic guar polymer has a charge density of from about 0.2 to about 2.2 meq/g, or from about 0.3 to about 2.0 meq/g, or from about 0.4 to about 1.8 meq/g; or from about 0.5 meq/g to about 1.7 meq/g.

The cationic guar polymer may have a weight average molecular weight of less than about 1.0 million g/mol, and has a charge density of about 0.1 meq/g to about 2.5 meq/g. In an embodiment, the cationic guar polymer has a weight average molecular weight of less than 950 thousand g/mol, or from about 10 thousand to about 900 thousand g/mol, or from about 25 thousand to about 900 thousand g/mol, or from about 50 thousand to about 900 thousand g/mol, or from about 100 thousand to about 900 thousand g/mol. from about 150 thousand to about 800 thousand g/mol. Alternatively, the cationic guar polymer may have a charge density of from about 0.2 to about 2.2 meq/g, or from about 0.3 to about 2.0 meq/g, or from about 0.4 to about 1.8 meq/g, or from about 0.5 meq/g to about 1.5 meq/g.

The shampoo composition can comprise from about 0.05% to less than about 1%, from about 0.05% to about 0.9%, from about 0.1% to about 0.8%, or from about 0.2% to about 0.7% of the one or more cationic polymers, by weight of the shampoo composition.

The cationic guar polymer may be formed from quaternary ammonium compounds. In an embodiment, the quaternary ammonium compounds for forming the cationic guar polymer conform to the general formula 1:

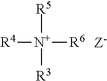

wherein where R³, R⁴ and R⁵ are methyl or ethyl groups; R⁶ is either an epoxyalkyl group of the general formula 2:

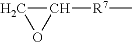

or R⁶ is a halohydrin group of the general formula 3:

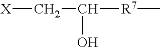

wherein R⁷ is a C₁ to C₃ alkylene; X is chlorine or bromine, and Z is an anion such as Cl—, Br—, I— or HSO₄—.

In an embodiment, the cationic guar polymer conforms to the general formula 4:

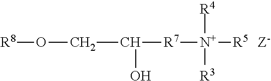

wherein R⁸ is guar gum; and wherein R⁴, R⁵, R⁶ and R⁷ are as defined above; and wherein Z is a halogen. In an embodiment, the cationic guar polymer conforms to formula 5:

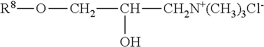

Suitable cationic guar polymers include cationic guar gum derivatives, such as guar hydroxypropyltrimonium chloride. The cationic guar polymer may be a guar hydroxypropyltrimonium chloride. Examples of guar hydroxypropyltrimonium chlorides include the Jaguar® series commercially available from Rhone-Poulenc Incorporated, for example Jaguar® C-500, commercially available from Rhodia. Jaguar® C-500 has a charge density of 0.8 meq/g and a molecular weight of 500,000 g/mol. Other suitable guar hydroxypropyltrimonium chloride include guar hydroxypropyltrimonium chloride which has a charge density of about 1.1 meq/g and a molecular weight of about 500,000 g/mol is available from ASI, a charge density of about 1.5 meq/g and a molecular weight of about 500,000 g/mole is available from ASI. Other suitable guar hydroxypropyltrimonium chloride include Hi-Care 1000, which has a charge density of about 0.7 meq/g and a molecular weight of about 600,000 g/mole and is available from Rhodia; N-Hance 3269 and N-Hance 3270, which has a charge density of about 0.7 meq/g and a molecular weight of about 425,000 g/mol and is available from ASIAquaCat CG518, has a charge density of about 0.9 meq/g, a molecular weight of about 50,000 g/mol, and is available from ASI. BF-13, which is a borate (boron) free guar of charge density of about 1.1 meq/g and molecular weight of about 800,000 and BF-17, which is a borate (boron) free guar of charge density of about 1.7 meq/g and M. W.t of about 800,000 both available from ASI.

The hair care compositions described herein may comprise a galactomannan polymer derivative having a mannose to galactose ratio of greater than 2:1 on a monomer to monomer basis, the galactomannan polymer derivative selected from the group consisting of a cationic galactomannan polymer derivative and an amphoteric galactomannan polymer derivative having a net positive charge. As used herein, the term “cationic galactomannan” refers to a galactomannan polymer to which a cationic group is added. The term “amphoteric galactomannan” refers to a galactomannan polymer to which a cationic group and an anionic group are added such that the polymer has a net positive charge.

Galactomannan polymers are present in the endosperm of seeds of the Leguminosae family. Galactomannan polymers are made up of a combination of mannose monomers and galactose monomers. The galactomannan molecule is a straight chain mannan branched at regular intervals with single membered galactose units on specific mannose units. The mannose units are linked to each other by means of β (1-4) glycosidic linkages. The galactose branching arises by way of an α (1-6) linkage. The ratio of mannose monomers to galactose monomers varies according to the species of the plant and also is affected by climate. Non Guar Galactomannan polymer derivatives of the present invention have a ratio of mannose to galactose of greater than 2:1 on a monomer to monomer basis. Suitable ratios of mannose to galactose can be greater than about 3:1, and the ratio of mannose to galactose can be greater than about 4:1. Analysis of mannose to galactose ratios is well known in the art and is typically based on the measurement of the galactose content.

The gum for use in preparing the non-guar galactomannan polymer derivatives is typically obtained as naturally occurring material such as seeds or beans from plants. Examples of various non-guar galactomannan polymers include but are not limited to Tara gum (3 parts mannose/1 part galactose), Locust bean or Carob (4 parts mannose/1 part galactose), and Cassia gum (5 parts mannose/1 part galactose).

The non-guar galactomannan polymer derivatives may have a molecular weight from about 1,000 to about1,000,000, and/or from about 5,000 to about900,000.

The hair care compositions may also include galactomannan polymer derivatives which have a cationic charge density from about 0.5 meq/g to about 7 meq/g. The galactomannan polymer derivatives may have a cationic charge density from about 1 meq/g to about 5 meq/g. The degree of substitution of the cationic groups onto the galactomannan structure should be sufficient to provide the requisite cationic charge density.

The galactomannan polymer derivative can be a cationic derivative of the non-guar galactomannan polymer, which is obtained by reaction between the hydroxyl groups of the polygalactomannan polymer and reactive quaternary ammonium compounds. Suitable quaternary ammonium compounds for use in forming the cationic galactomannan polymer derivatives include those conforming to the general formulas 1-5, as defined above.

Cationic non-guar galactomannan polymer derivatives formed from the reagents described above are represented by the general formula 6:

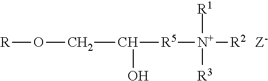

wherein R is the gum. The cationic galactomannan derivative can be a gum hydroxypropyltrimethylammonium chloride, which can be more specifically represented by the general formula 7:

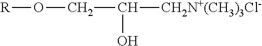

Alternatively the galactomannan polymer derivative can be an amphoteric galactomannan polymer derivative having a net positive charge, obtained when the cationic galactomannan polymer derivative further comprises an anionic group.

The cationic non-guar galactomannan can have a ratio of mannose to galactose is greater than about 4:1, a molecular weight of about 50,000 g/mol to about 1,000,000 g/mol, and/or from about 100,000 g/mol to about 900,000 g/mol and a cationic charge density from about 1 meq/g to about 5 meq/g, and/or from 2 meq/g to about 4 meq/g and can also be derived from a cassia plant.

The hair care compositions may comprise at least about 0.05% of a galactomannan polymer derivative by weight of the composition, alternatively from about 0.05% to about 2%, by weight of the composition, of a galactomannan polymer derivative.

The hair care compositions may comprise water-soluble cationically modified starch polymers. As used herein, the term “cationically modified starch” refers to a starch to which a cationic group is added prior to degradation of the starch to a smaller molecular weight, or wherein a cationic group is added after modification of the starch to achieve a desired molecular weight. The definition of the term “cationically modified starch” also includes amphoterically modified starch. The term “amphoterically modified starch” refers to a starch hydrolysate to which a cationic group and an anionic group are added.

The hair care compositions may comprise cationically modified starch polymers at a range of about 0.01% to about 10%, and/or from about 0.05% to about 5%, by weight of the composition.

The cationically modified starch polymers disclosed herein have a percent of bound nitrogen of from about 0.5% to about 4%.

The cationically modified starch polymers for use in the hair care compositions can have a molecular weight about 50,000 g/mol to about 1,000,000 g/mol and/or from about 100,000 g/mol to about 1,000,000 g/mol.

The hair care compositions may include cationically modified starch polymers which have a charge density of from about 0.2 meq/g to about 5 meq/g, and/or from about 0.2 meq/g to about 2 meq/g. The chemical modification to obtain such a charge density includes, but is not limited to, the addition of amino and/or ammonium groups into the starch molecules. Non-limiting examples of these ammonium groups may include substituents such as hydroxypropyl trimmonium chloride, trimethylhydroxypropyl ammonium chloride, dimethylstearylhydroxypropyl ammonium chloride, and dimethyldodecylhydroxypropyl ammonium chloride. See Solarek, D. B., Cationic Starches in Modified Starches: Properties and Uses, Wurzburg, O. B., Ed., CRC Press, Inc., Boca Raton, Fla. 1986, pp 113-125. The cationic groups may be added to the starch prior to degradation to a smaller molecular weight or the cationic groups may be added after such modification.

The cationically modified starch polymers generally have a degree of substitution of a cationic group from about 0.2 to about 2.5. As used herein, the “degree of substitution” of the cationically modified starch polymers is an average measure of the number of hydroxyl groups on each anhydroglucose unit which is derivatized by substituent groups. Since each anhydroglucose unit has three potential hydroxyl groups available for substitution, the maximum possible degree of substitution is 3. The degree of substitution is expressed as the number of moles of substituent groups per mole of anhydroglucose unit, on a molar average basis. The degree of substitution may be determined using proton nuclear magnetic resonance spectroscopy (“.sup.1H NMR”) methods well known in the art. Suitable .sup.1H NMR techniques include those described in “Observation on NMR Spectra of Starches in Dimethyl Sulfoxide, Iodine-Complexing, and Solvating in Water-Dimethyl Sulfoxide”, Qin-Ji Peng and Arthur S. Perlin, Carbohydrate Research, 160 (1987), 57-72; and “An Approach to the Structural Analysis of Oligosaccharides by NMR Spectroscopy”, J. Howard Bradbury and J. Grant Collins, Carbohydrate Research, 71, (1979), 15-25.

The source of starch before chemical modification can be chosen from a variety of sources such as tubers, legumes, cereal, and grains. Non-limiting examples of this source starch may include corn starch, wheat starch, rice starch, waxy corn starch, oat starch, cassaya starch, waxy barley, waxy rice starch, glutenous rice starch, sweet rice starch, amioca, potato starch, tapioca starch, oat starch, sago starch, sweet rice, or mixtures thereof.

The cationically modified starch polymers can be selected from degraded cationic maize starch, cationic tapioca, cationic potato starch, and mixtures thereof. Alternatively, the cationically modified starch polymers are cationic corn starch and cationic tapioca.

The starch, prior to degradation or after modification to a smaller molecular weight, may comprise one or more additional modifications. For example, these modifications may include cross-linking, stabilization reactions, phosphorylations, and hydrolyzations. Stabilization reactions may include alkylation and esterification.

The cationically modified starch polymers may be incorporated into the composition in the form of hydrolyzed starch (e.g., acid, enzyme, or alkaline degradation), oxidized starch (e.g., peroxide, peracid, hypochlorite, alkaline, or any other oxidizing agent), physically/mechanically degraded starch (e.g., via the thermo-mechanical energy input of the processing equipment), or combinations thereof.

An optimal form of the starch is one which is readily soluble in water and forms a substantially clear (% Transmittance.gtoreq.80 at 600 nm) solution in water. The transparency of the composition is measured by Ultra-Violet/Visible (UV/VIS) spectrophotometry, which determines the absorption or transmission of UV/VIS light by a sample, using a Gretag Macbeth Colorimeter Color i 5 according to the related instructions. A light wavelength of 600 nm has been shown to be adequate for characterizing the degree of clarity of cosmetic compositions.

Also suitable for use in the hair care composition described herein are nonionic modified starches that can be further derivatized to a cationically modified starch as is known in the art. Other suitable modified starch starting materials may be quaternized to produce the cationically modified starch polymer suitable for use in hair care compositions.

Starch Degradation Procedure: a starch slurry can be prepared by mixing granular starch in water. The temperature is raised to about 35° C. An aqueous solution of potassium permanganate is then added at a concentration of about 50 ppm based on starch. The pH is raised to about 11.5 with sodium hydroxide and the slurry is stirred sufficiently to prevent settling of the starch. Then, about a 30% solution of hydrogen peroxide diluted in water is added to a level of about 1% of peroxide based on starch. The pH of about 11.5 is then restored by adding additional sodium hydroxide. The reaction is completed over about a 1 to about 20 hour period. The mixture is then neutralized with dilute hydrochloric acid. The degraded starch is recovered by filtration followed by washing and drying. The hair care composition can comprise a cationic copolymer of an acrylamide monomer and a cationic monomer, wherein the copolymer has a charge density of from about 1.0 meq/g to about 3.0 meq/g. The cationic copolymer can be a synthetic cationic copolymer of acrylamide monomers and cationic monomers.

The cationic copolymer can comprise:
(i) an acrylamide monomer of the following Formula AM:

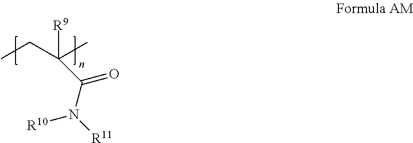

where R⁹ is H or C₁₋₄ alkyl; and R¹⁰ and R¹¹ are independently selected from the group consisting of H, C₁₋₄ alkyl, CH₂OCH₃, CH₂OCH₂CH(CH₃)₂, and phenyl, or together are C₃₋₆cycloalkyl; and
(ii) a cationic monomer conforming to Formula CM:

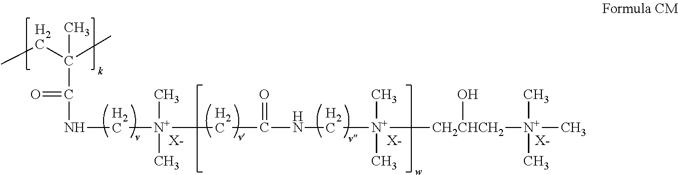

where k=1, each of v, v′, and v″ is independently an integer of from 1 to 6, w is zero or an integer of from 1 to 10, and X⁻ is an anion.

The cationic monomer can conform to Formula CM and where k=1, v=3 and w=0, z=1 and X⁻ is Cl⁻ to form the following structure:

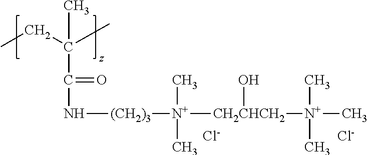

The above structure may be referred to as diquat. Alternatively, the cationic monomer can conform to Formula CM and wherein v and v″ are each 3, v′=1, w=1, y=1 and X⁻ is Cl⁻, such as:

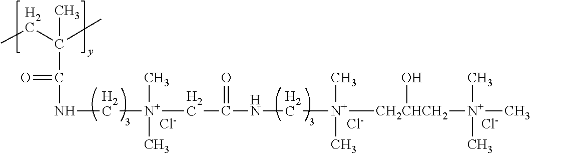

The above structure may be referred to as triquat.

Suitable acrylamide monomer include, but are not limited to, either acrylamide or methacrylamide.

In an alternative embodiment, the cationic copolymer is of an acrylamide monomer and a cationic monomer, wherein the cationic monomer is selected from the group consisting of: dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide; ethylenimine, vinylamine, 2-vinylpyridine, 4-vinylpyridine; trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride, and mixtures thereof.

The cationic copolymer can comprise a cationic monomer selected from the group consisting of: cationic monomers include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, and mixtures thereof.

The cationic copolymer can be water-soluble. The cationic copolymer is formed from (1) copolymers of (meth)acrylamide and cationic monomers based on (meth)acrylamide, and/or hydrolysis-stable cationic monomers, (2) terpolymers of (meth)acrylamide, monomers based on cationic (meth)acrylic acid esters, and monomers based on (meth)acrylamide, and/or hydrolysis-stable cationic monomers. Monomers based on cationic (meth)acrylic acid esters may be cationized esters of the (meth)acrylic acid containing a quaternized N atom. In an embodiment, cationized esters of the (meth)acrylic acid containing a quaternized N atom are quaternized dialkylaminoalkyl (meth)acrylates with C1 to C3 in the alkyl and alkylene groups. Suitable cationized esters of the (meth)acrylic acid containing a quaternized N atom can be selected from the group consisting of: ammonium salts of dimethylaminomethyl (meth)acrylate, dimethylaminoethyl (meth)acrylate, dimethylaminopropyl (meth)acrylate, diethylaminomethyl (meth)acrylate, diethylaminoethyl (meth)acrylate; and diethylaminopropyl (meth)acrylate quaternized with methyl chloride. In an embodiment, the cationized esters of the (meth)acrylic acid containing a quaternized N atom is dimethylaminoethyl acrylate, which is quaternized with an alkyl halide, or with methyl chloride or benzyl chloride or dimethyl sulfate (ADAME-Quat). the cationic monomer when based on (meth)acrylamides can be quaternized dialkylaminoalkyl(meth)acrylamides with C1 to C3 in the alkyl and alkylene groups, or dimethylaminopropylacrylamide, which is quaternized with an alkyl halide, or methyl chloride or benzyl chloride or dimethyl sulfate.

Suitable cationic monomer based on a (meth)acrylamide include quaternized dialkylaminoalkyl(meth)acrylamide with C1 to C3 in the alkyl and alkylene groups. The cationic monomer based on a (meth)acrylamide can be dimethylaminopropylacrylamide, which is quaternized with an alkyl halide, especially methyl chloride or benzyl chloride or dimethyl sulfate.

The cationic monomer can be a hydrolysis-stable cationic monomer. Hydrolysis-stable cationic monomers can be, in addition to a dialkylaminoalkyl(meth)acrylamide, all monomers that can be regarded as stable to the OECD hydrolysis test. The cationic monomer can be hydrolysis-stable and the hydrolysis-stable cationic monomer can be selected from the group consisting of: diallyldimethylammonium chloride and water-soluble, cationic styrene derivatives. The cationic copolymer can be a terpolymer of acrylamide, 2-dimethylammoniumethyl (meth)acrylate quaternized with methyl chloride (ADAME-Q) and 3-dimethylammoniumpropyl(meth)acrylamide quaternized with methyl chloride (DIMAPA-Q). The cationic copolymer can be formed from acrylamide and acrylamidopropyltrimethylammonium chloride, wherein the acrylamidopropyltrimethylammonium chloride has a charge density of from about 1.0 meq/g to about 3.0 meq/g.

The cationic copolymer can have a charge density of from about 1.1 meq/g to about 2.5 meq/g, or from about 1.1 meq/g to about 2.3 meq/g, or from about 1.2 meq/g to about 2.2 meq/g, or from about 1.2 meq/g to about 2.1 meq/g, or from about 1.3 meq/g to about 2.0 meq/g, or from about 1.3 meq/g to about 1.9 meq/g.

The cationic copolymer can have a molecular weight from about 10 thousand g/mol to about 1 million g/mol, or from about 25 thousand g/mol to about 1 million g/mol, or from about 50 thousand g/mol to about 1 million g/mol, or from about 100 thousand g/mol to about 1.0 million g/mol, or from about 150 thousand g/mol to about 1.0 million g/mol.

(a) Cationic Synthetic Polymers

The hair care composition can comprise a cationic synthetic polymer that may be formed from
i) one or more cationic monomer units, and optionally
ii) one or more monomer units bearing a negative charge, and/or
iii) a nonionic monomer,
   wherein the subsequent charge of the copolymer is positive. The ratio of the three types of monomers is given by “m”, “p” and “q” where “m” is the number of cationic monomers, “p” is the number of monomers bearing a negative charge and “q” is the number of nonionic monomers

The cationic polymers can be water soluble or dispersible, non-crosslinked, and synthetic cationic polymers having the following structure:

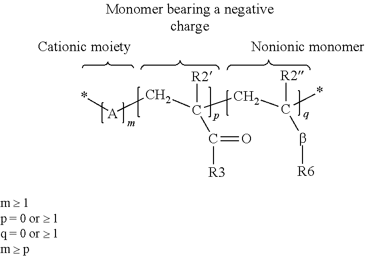

where A, may be one or more of the following cationic moieties:

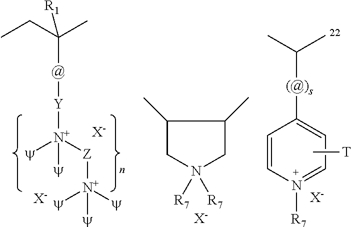

where @=amido, alkylamido, ester, ether, alkyl or alkylaryl;
where Y═C1-C22 alkyl, alkoxy, alkylidene, alkyl or aryloxy;
where ψ=C1-C22 alkyl, alkyloxy, alkyl aryl or alkyl arylox;.
where Z═C1-C22 alkyl, alkyloxy, aryl or aryloxy;
where R1=H, C1-C4 linear or branched alkyl;
where s=0or 1,n=0or 1;
where T and R7=C1-C22 alkyl; and
where X—=halogen, hydroxide, alkoxide, sulfate or alkylsulfate.

Where the monomer bearing a negative charge is defined by R2′=H, C1-C4 linear or branched alkyl and R3 as:

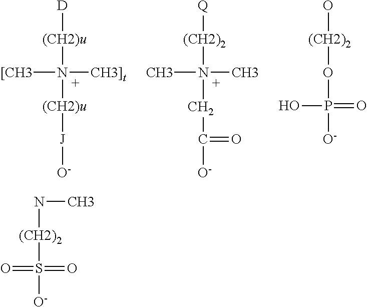

where D=O, N, or S;
where Q=NH₂ or O;
where u=1-6;
where t=0-1; and
where J=oxygenated functional group containing the following elements P, S, C.
   Where the nonionic monomer is defined by R2″=H, C1-C4 linear or branched alkyl, R6=linear or branched alkyl, alkyl aryl, aryl oxy, alkyloxy, alkylaryl oxy and β is defined as

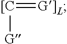

and
where G′ and G″ are, independently of one another, O, S or N—H and L=0 or 1.

Examples of cationic monomers include aminoalkyl (meth)acrylates, (meth)aminoalkyl (meth)acrylamides; monomers comprising at least one secondary, tertiary or quaternary amine function, or a heterocyclic group containing a nitrogen atom, vinylamine or ethylenimine; diallyldialkyl ammonium salts; their mixtures, their salts, and macromonomers deriving from therefrom.

Further examples of cationic monomers include dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide, ethylenimine, vinylamine, 2-vinylpyridine, 4-vinylpyridine, trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride.

Suitable cationic monomers include those which comprise a quaternary ammonium group of formula —NR₃⁺, wherein R, which is identical or different, represents a hydrogen atom, an alkyl group comprising 1 to 10 carbon atoms, or a benzyl group, optionally carrying a hydroxyl group, and comprise an anion (counter-ion). Examples of anions are halides such as chlorides, bromides, sulphates, hydrosulphates, alkylsulphates (for example comprising 1 to 6 carbon atoms), phosphates, citrates, formates, and acetates.

Suitable cationic monomers include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride.

Additional suitable cationic monomers include trimethyl ammonium propyl (meth)acrylamido chloride.

Examples of monomers bearing a negative charge include alpha ethylenically unsaturated monomers comprising a phosphate or phosphonate group, alpha ethylenically unsaturated monocarboxylic acids, monoalkylesters of alpha ethylenically unsaturated dicarboxylic acids, monoalkylamides of alpha ethylenically unsaturated dicarboxylic acids, alpha ethylenically unsaturated compounds comprising a sulphonic acid group, and salts of alpha ethylenically unsaturated compounds comprising a sulphonic acid group.

Suitable monomers with a negative charge include acrylic acid, methacrylic acid, vinyl sulphonic acid, salts of vinyl sulfonic acid, vinylbenzene sulphonic acid, salts of vinylbenzene sulphonic acid, alpha-acrylamidomethylpropanesulphonic acid, salts of alpha-acrylamidomethylpropanesulphonic acid, 2-sulphoethyl methacrylate, salts of 2-sulphoethyl methacrylate, acrylamido-2-methylpropanesulphonic acid (AMPS), salts of acrylamido-2-methylpropanesulphonic acid, and styrenesulphonate (SS).

Examples of nonionic monomers include vinyl acetate, amides of alpha ethylenically unsaturated carboxylic acids, esters of an alpha ethylenically unsaturated monocarboxylic acids with an hydrogenated or fluorinated alcohol, polyethylene oxide (meth)acrylate (i.e. polyethoxylated (meth)acrylic acid), monoalkylesters of alpha ethylenically unsaturated dicarboxylic acids, monoalkylamides of alpha ethylenically unsaturated dicarboxylic acids, vinyl nitriles, vinylamine amides, vinyl alcohol, vinyl pyrolidone, and vinyl aromatic compounds.

Suitable nonionic monomers include styrene, acrylamide, methacrylamide, acrylonitrile, methylacrylate, ethylacrylate, n-propylacrylate, n-butylacrylate, methylmethacrylate, ethylmethacrylate, n-propylmethacrylate, n-butylmethacrylate, 2-ethyl-hexyl acrylate, 2-ethyl-hexyl methacrylate, 2-hydroxyethylacrylate and 2-hydroxyethylmethacrylate.

The anionic counterion (X—) in association with the synthetic cationic polymers may be any known counterion so long as the polymers remain soluble or dispersible in water, in the hair care composition, or in a coacervate phase of the hair care composition, and so long as the counterions are physically and chemically compatible with the essential components of the hair care composition or do not otherwise unduly impair product performance, stability or aesthetics. Non limiting examples of such counterions include halides (e.g., chlorine, fluorine, bromine, iodine), sulfate and methylsulfate.

The concentration of the cationic polymers ranges about 0.025% to about 5%, from about 0.1% to about 3%, and/or from about 0.2% to about 1%, by weight of the hair care composition.

Suitable cationic cellulose polymers are salts of hydroxyethyl cellulose reacted with trimethyl ammonium substituted epoxide, referred to in the industry (CTFA) as Polyquaternium 10 and available from Dow/Amerchol Corp. (Edison, N.J., USA) in their Polymer LR, JR, and KG series of polymers. Other suitable types of cationic cellulose include the polymeric quaternary ammonium salts of hydroxyethyl cellulose reacted with lauryl dimethyl ammonium-substituted epoxide referred to in the industry (CTFA) as Polyquaternium 24. These materials are available from Dow/Amerchol Corp. under the tradename Polymer LM-200. Other suitable types of cationic cellulose include the polymeric quaternary ammonium salts of hydroxyethyl cellulose reacted with lauryl dimethyl ammonium-substituted epoxide and trimethyl ammonium substituted epoxide referred to in the industry (CTFA) as Polyquaternium 67. These materials are available from Dow/Amerchol Corp. under the tradename SoftCAT Polymer SL-5, SoftCAT Polymer SL-30, Polymer SL-60, Polymer SL-100, Polymer SK-L, Polymer SK-M, Polymer SK-MH, and Polymer SK-H.

D. Viscosity Reducing Agent

The shampoo composition may comprise from about 1% to about 10%, alternatively from about 3.25% to about 9%, alternatively from about 3.5% to about 8%, alternatively from about 2% to about 7%, and alternatively from about 4% to about 7% of one or more viscosity reducing agents, by weight of the shampoo composition.

The viscosity reducing agents may have a partition dispersion coefficient of from about −5 to about −0.7, alternatively from about −4.6 to about −0.85, alternatively from about −4.5 to about −0.9, alternatively from about −3.1 to about −0.7, and alternatively from about −3 to about −0.85. The viscosity reducing agents may have a partition dispersion coefficient of from about −4.6 to about −1.9, alternatively from about −4.5 to about −2, wherein the one or more viscosity reducing agents has at least 2 polar groups, or has 1 polar group and less than 5 acyclic sp³ hybridized carbon atoms that are connected to each other in a contiguous group. The viscosity reducing agents may have a partition dispersion coefficient of from about −4.6 to about −1.9, alternatively from about −4.5 to about −2, wherein the one or more viscosity reducing agents has 2 to 4 polar groups, or has 1 polar group and 1 to 3 acyclic sp³ hybridized carbon atoms that are connected to each other in a contiguous group. The viscosity reducing agents may have a partition dispersion coefficient of from about −4.6 to about −1.9, alternatively from about −4.5 to about −2, wherein the one or more viscosity reducing agents has 2 to 4 polar groups, or has 1 polar group and 2 acyclic sp³ hybridized carbon atoms that are connected to each other in a contiguous group. The viscosity reducing agents may provide unexpected viscosity reduction when used in the hair care composition described herein.

The viscosity reducing agents may have a partition dispersion coefficient of from about 0.05 to about 5.1, alternatively from about 0.08 to about 4.5, alternatively from about 0.09 to about 4.4, alternatively from about 0.05 to about 2.0, alternatively from about 0.08 to about 1.8, alternatively from about 0.09 to about 1.7, and alternatively from about 0.095 to about 1.68. The viscosity reducing agents may provide unexpected viscosity reduction when used in the hair care composition described herein.

The partition dispersion coefficient (PDC) is defined by the following equation:

PDC=logP−0.3001*(δD)²+10.362*δD−93.251

wherein logP is the octanol water partitioning coefficient as computed by the Consensus algorithm implemented in ACD/Percepta version 14.02 by Advanced Chemistry Development, Inc. (ACD/Labs, Toronto, Canada), and wherein δD is the Hansen solubility dispersion parameter in (MPa)^{1/2} computed using Steven Abbott and Hiroshi Yamamoto's “HSPIP—Hansen Solubility Parameters in Practice” program, 4^{th} Edition, version 4.1.07.

The viscosity reducing agents may be organic compounds comprising 0 polar groups, alternatively 1 polar group, alternatively at least 1 polar group, alternatively 2 to 4 polar groups, and alternative alternatively at least 2 polar groups. The polar groups may be selected from the group consisting of alcohols, aldehydes, esters, lactones, coumarins, ethers, ketones, phenol, phenyl, oxides, alkenyl, alkynyl, and combinations thereof. The polar groups may include a carbon-carbon double bond or one or more atoms selected from the group consisting of oxygen, sulfur, phosphorus, chlorine, bromine, and combinations thereof. The viscosity reducing agents may have a molecular weight of between 100 daltons and 300 daltons, alternatively from about 125 daltons to about 300 daltons. Additionally, the viscosity reducing agents may have a water solubility at between 23 and 25 degrees Celsius of from about 900 to 50,000 mg/L.

The viscosity reducing agents may be selected from the group consisting of raspberry ketone, triethyl citrate, 5-methyl-3-heptanone oxime, hydroxycitronellal, camphor gum, 2-isopropyl-5-methyl-2-hexenal, eucalyptol, 1,1-dimethoxyoctane, isobutyl hexanoate, dihyro iso jasmonate, and combinations thereof. Alternatively, the viscosity reducing agents may be selected from the group consisting of raspberry ketone, triethyl citrate, hydroxycitronellal, ethanol, dipropylene glycol, and combinations thereof.

The viscosity reducing agents may be selected from the group consisting of veloutone, isoamyl salicylate, gamma-terpinene, linalyl iso butyrate, alpha-terpinene, limonene, dipentene, geranyl phenyl acetate, iso propyl myristate, hexadecane, and combinations thereof. Alternatively, the counteracting additive may be selected from the group consisting of veloutone, gamma-terpinene, linalyl iso butyrate, alpha-terpinene, limonene, dipentene, geranyl phenyl acetate, iso propyl myristate, hexadecane, and combinations thereof. Alternatively, the counteracting additive may be selected from the group consisting of veloutone, isoamyl salicylate, gamma-terpinene, linalyl iso butyrate, alpha-terpinene, limonene, dipentene, geranyl phenyl acetate, and combinations thereof.

E. Shampoo Viscosity

The shampoo composition may have a kinematic viscosity of from about 10 cSt to about 500 cSt, alternatively from about 15 cSt to about 400 cSt, alternatively from about 20 cSt to about 300 cSt, alternatively from about 25 cSt to about 250 cSt, and alternatively from about 30 cSt to about 250 cSt. In an embodiment, the shampoo composition may have a liquid phase viscosity of from about 1 centipoise to about 3,000 centipoise, alternatively from about 1 centipoise to about 2,500 centipoise, alternatively from about 1 centipoise to about 2,000 centipoise, alternatively from about 5 centipoise to about 1,500 centipoise, and alternatively from about 10 centipoise to about 1,200 centipoise. In another embodiment, the shampoo composition may have a liquid phase viscosity of from about 1 centipoise to about 15,000 centipoise, alternatively from about 1,000 centipoise to about 12,500 centipoise, alternatively from about 2,000 centipoise to about 10,000 centipoise, and alternatively from about 3,000 centipoise to about 7,500 centipoise. The hair composition viscosity values may be measured using a TA Instruments AR-G2 Rheometer with a concentric cylinder attachment at a shear rate of 100 reciprocal seconds at 25° C.

### Concentrated Conditioner Composition

A. Oil Deposition Purity

The method of treating hair comprises dispensing the concentrated conditioner composition described herein from the aerosol foam dispenser as a dosage of foam. The foam may comprise a oil deposition purity of from about 40% to about 100%, alternatively from about 50% to about 100%, alternatively from about 60% to about 100%, alternatively from about 70% to about 100%, and alternatively from about 80% to about 100%, after applying the foam to the hair and rinsing the foam from the hair.

Deposition Purity is determined by the ratio of oil deposited per weight of hair to the total deposition of other ingredients per weight of hair. Oil is determined by either extraction or digestion of the hair followed by an analysis with quantitative elemental techniques such as ICP in the case of silicones or total silicon and converting to silicone based on the % of silicon in the silicone by weight. The total deposition may be determined by the sum of separate deposition measurements or by a Single Inclusive Measurement of total deposition. The separate deposition measurements may include but are not limited to: fatty alcohols, EGDS, quaternized agents, oils and silicone. Typically these measurements involve extracting the hair then separating the ingredients of interest with chromatography and quantifying with an externally calibration based on test solution concentration. The Single Inclusive Measurement of total deposition is gravimetric. The hair is thoroughly extracted and the residue determined by weighing the dissolved residue in the extract after evaporating the solvent. This residue contains both deposited ingredients and naturally occurring extractable compounds from the hair (primarily lipids). The naturally occurring extractable compounds are quantified and subtracted from the total. These include: fatty acids, squalene, cholesterol, ceramides, wax esters, triglycerides and sterol esters. The method of quantitation is similar to the deposition measurements. Other supporting evidence of Deposition Purity may include spectroscopic or topography mapping of the hair surface.

B. Oils

The concentrated conditioner composition may comprise from about 4% to about 22%, alternatively from about 5% to about 20%, alternatively from about 8% to about 18%, and alternatively from about 10% to about 14% of one or more oils, by weight of the concentrated conditioner composition. The one or more oils may be selected from the group consisting of silicones, natural oils, organic conditioner materials, and combinations thereof. The particle size of the one or more oils may be from about 1 nm to about 300 nm, alternatively from about 1 nm to about 100 nm, alternatively from about 5 nm to about 80 nm, alternatively from about 10 nm to about 60 nm, and alternatively from about 12 nm to about 50 nm.

The particle size of the one or more oils may be measured by dynamic light scattering (DLS). A Malvern Zetasizer Nano ZEN3600 system (www.malvern.com) using He-Ne laser 633 nm may be used used for the measurement at 25° C.

The autocorrelation function may be analyzed using the Zetasizer Software provided by Malvern Instruments, which determines the effective hydrodynamic radius, using the Stokes-Einstein equation:

$$D = \frac{{k}_{B} T}{6 πη R}$$

wherein k_{B} is the Boltzmann Constant, T is the absolute temperature, η is the viscosity of the medium, D is the mean diffusion coefficient of the scattering species, and R is the hydrodynamic radius of particles.

Particle size (i.e. hydrodynamic radius) may be obtained by correlating the observed speckle pattern that arises due to Brownian motion and solving the Stokes-Einstein equation, which relates the particle size to the measured diffusion constant, as is known in the art.

For each sample, 3 measurements may be made and Z-average values may be reported as the particle size.

In an embodiment, the one or more oils may be in the form of a nanoemulsion. The nanoemulsion may comprise any oils suitable for application to the skin and/or hair.

In an embodiment, the one or more silicones may include in their molecular structure polar functional groups such as Si—OH (present in dimethiconols), primary amines, secondary amines, tertiary amines, and quaternary ammonium salts. The one or more silicones may be selected from the group consisting of aminosilicones, pendant quaternary ammonium silicones, terminal quaternary ammonium silicones, amino polyalkylene oxide silicones, quaternary ammonium polyalkylene oxide silicones, and amino morpholino silicones.

The one or more silicones may comprise:
(a) at least one aminosilicone corresponding to formula (V):

R′ₐG₃₋ₐ-Si(OSiG₂)ₙ—(OSiG_{b}R′_{2-b})ₘ—O—SiG₃₋ₐR′ₐ (I)
in which:
G is chosen from a hydrogen atom, a phenyl group, OH group, and C₁-C₈ alkyl groups, for example methyl,
a is an integer ranging from 0 to 3, and in one embodiment a is 0,
b is chosen from 0 and 1, and in one embodiment b is 1,
m and n are numbers such that the sum (n+m) can range for example from 1 to 2 000, such as for example from 50 to 150, wherein n can be for example chosen from numbers ranging from 0 to 1 999, such as for example from 49 to 149, and wherein m can be chosen from numbers ranging for example from 1 to 2 000, such as for example from 1 to 10;
R′ is a monovalent group of formula —C_{q}H_{2q}L in which q is a number from 2 to 8 and L is an optionally quaternized amine group chosen from the groups:
—NR″—CH₂—CH₂—N′(R¹)₂,
—N(R″)₂,
—N⁺(R″)₃A⁻,
—N⁺H(R″)₂A⁻,
—N⁺H₂(R″)A⁻, and
—N(R″)—CH₂—CH₂—N⁺R″H₂A⁻,
   in which R″ can be chosen from a hydrogen atom, phenyl groups, benzyl groups, and saturated monovalent hydrocarbon-based groups, such as for example an alkyl group comprising from 1 to 20 carbon atoms, and A⁻ is chosen from halide ions such as, for example, fluoride, chloride, bromide and iodide.

In an embodiment, the one or more silicones may include those corresponding to formula (1) wherein a=0, G=methyl, m and n are numbers such that the sum (n+m) can range for example from 1 to 2 000, such as for example from 50 to 150, wherein n can be for example chosen from numbers ranging from 0 to 1 999, such as for example from 49 to 149, and wherein m can be chosen from numbers ranging for example from 1 to 2 000, such as for example from 1 to 10; and L is —N(CH₃)₂ or —NH₂, alternatively —NH₂.
Additional said at least one aminosilicone of the invention include:
(b) pendant quaternary ammonium silicones of formula (VII):

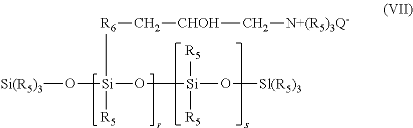

in which:
R₅ is chosen from monovalent hydrocarbon-based groups comprising from 1 to 18 carbon atoms, such as C₁-C₁₈ alkyl groups and C₂-C₁₈ alkenyl groups, for example methyl;
R₆ is chosen from divalent hydrocarbon-based groups, such as divalent C₁-C₁₈ alkylene groups and divalent C₁-C₁₈ alkylenoxy groups, for example C₁-C₈ alkylenoxy groups, wherein said R₆ is bonded to the Si by way of an SiC bond;
Q⁻ is an anion that can be for example chosen from halide ions, such as chloride, and organic acid salts (such as acetate);
r is an average statistical value ranging from 2 to 20, such as from 2 to 8;
s is an average statistical value ranging from 20 to 200, such as from 20 to 50.

Such aminosilicones are described more particularly in U.S. Pat. No. 4,185,087, the disclosure of which is incorporated by reference herein.

A silicone which falls within this class is the silicone sold by the company Union Carbide under the name “Ucar Silicone ALE 56”.

Further examples of said at least one aminosilicone include:
c) quaternary ammonium silicones of formula (VIIb):

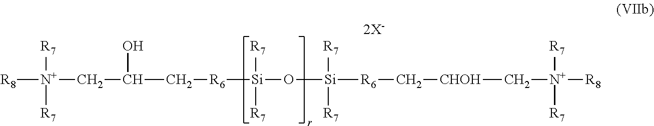

in which:
groups R_{7,} which may be identical or different, are each chosen from monovalent hydrocarbon-based groups comprising from 1 to 18 carbon atoms, such as C₁-C₁₈ alkyl groups, for example methyl, C₂-C₁₈ alkenyl groups, and rings comprising 5 or 6 carbon atoms;
R₆ is chosen from divalent hydrocarbon-based groups, such as divalent C₁-C₁₈ alkylene groups and divalent C₁-C₁₈alkylenoxy, for example C₁-C₈, group connected to the Si by an SiC bond;
R_{8,} which may be identical or different, represent a hydrogen atom, a monovalent hydrocarbon-based group comprising from 1 to 18 carbon atoms, and in particular a C₁-C₁₈ alkyl group, a C₂-C₁₈ alkenyl group or a group —R₆—NHCOR₇;
X⁻ is an anion such as a halide ion, in particular chloride, or an organic acid salt (acetate, etc.);
r represents an average statistical value from 2 to 200 and in particular from 5 to 100.
Such silicones are described, for example, in application EP-A-0 530 974, the disclosure of which is incorporated by reference herein.
Silicones falling within this class are the silicones sold by the company Goldschmidt under the names Abil Quat 3270, Abil Quat 3272 and Abil Quat 3474.
Further examples of said at least one aminosilicone include:
d) quaternary ammonium and polyalkylene oxide silicones
wherein the quaternary nitrogen groups are located in the polysiloxane backbone, at the termini, or both.
Such silicones are described in PCT Publication No. WO 2002/010257, the disclosure of which is incorporated by reference herein.
Siliciones falling within this class are the silicones sold by the company Momentive under the names Silsoft Q. . . .
(e) Aminofunctional silicones having morpholino groups of formula (V):

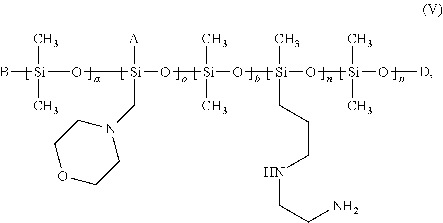

in which
A denotes a structural unit (I), (II), or (III) bound via —O—

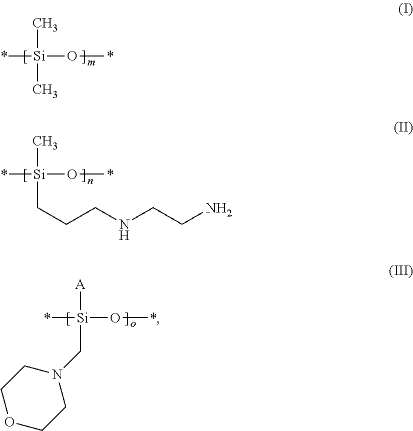

or an oligomeric or polymeric residue, bound via —O—, containing structural units of formulas (I), (II), or (III), or half of a connecting oxygen atom to a structural unit (III), or denotes —OH,
. denotes a bond to one of the structural units (I), (II), or (III), or denotes a terminal group B (Si-bound) or D (O-bound),
B denotes an —OH, —O—Si(CH₃)₃, —O—Si(CH₃)₂OH, —O—Si(CH₃)₂OCH₃ group,
D denotes an —H, —Si(CH₃)₃, —Si(CH₃)₂OH, —Si(CH₃)₂OCH₃ group,
a, b, and c denote integers between 0 and 1000, with the provision that a+b+c>0,
m, n, and o denote integers between 1 and 1000.

Aminofunctional silicones of this kind bear the INCI name: Amodimethicone/Morpholinomethyl Silsesquioxane Copolymer. A particularly suitable amodimethicone is the product having the commercial name Wacker Belsil® ADM 8301E.

Examples of such silicones are available from the following suppliers:
offered by the company Dow Corning:
   Fluids: 2-8566, AP 6087, AP 6088, DC 8040 Fluid, fluid 8822A DC, DC 8803 & 8813 polymer, 7-6030, AP-8104, AP 8201;
   Emulsions: CE-8170 AF Micro Emulsion, 2-8177, 2-8194 Microemulsion, 9224 Emulsion, 939, 949, 959, DC 5-7113 Quat Microemulsion, DC 5-7070 Emulsion, DC CE-8810, CE 8401 Emulsion, CE 1619, Dow Corning Toray SS-3551, Dow Corning Toray SS-3552;
offered by the company Wacker:
   Wacker Belsil ADM 652, ADM 656, 1100, 1600, 1650 (fluids) ADM 6060 (linear amodimethicone) emulsion; ADM 6057 E (branched amodimethicone) emulsion; ADM 8020 VP (micro emulsion); SLM 28040 (micro emulsion);
offered by the Company Momentive:
   Silsoft 331, SF1708, SME 253 & 254 (emulsion), SM2125 (emulsion), SM 2658 (emulsion), Silsoft Q (emulsion)
offered by the company Shin-Etsu:
   KF-889, KF-8675, KF-8004, X-52-2265 (emulsion);
offered by the Company Siltech Silicones:
   Siltech E-2145, E-Siltech 2145-35;
offered by the company Evonik Industries:
   Abil T Quat 60th

Some non-limiting examples of aminosilicones include the compounds having the following INCI names: Silicone Quaternium-1, Silicone Quaternium-2, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, Silicone Quaternium-16, Silicone Quaternium-17, Silicone Quaternium-18, Silicone Quaternium-20, Silicone Quaternium-21, Silicone Quaternium-22, Quaternium-80, as well as Silicone Quaternium-2 Panthenol Succinate and Silicone Quaternium-16/Glycidyl Dimethicone Crosspolymer.

In an embodiment, the aminosilicones can be supplied in the form of a nanoemulsion and include MEM 9049, MEM 8177, MEM 0959, MEM 8194, SME 253, and Silsoft Q.

In an embodiment, the one or more silicones may include dimethicones, and/or dimethiconols. The dimethiconols are hydroxyl terminated dimethylsilicones represented by the general chemical formulas

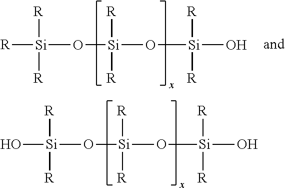

wherein R is an alkyl group (preferably R is methyl or ethyl, more preferably methyl) and x is an integer up to about 500, chosen to achieve the desired molecular weight. Commercial dimethiconols typically are sold as mixtures with dimethicone or cyclomethicone (e.g.,Dow Coming® 1401, 1402, and 1403 fluids).

In an embodiment, the one or more oils include low melting point non-silicone oils having a melting point of from about −50 degrees Celsius to about 38 degrees Celsius, alternatively from about −45 degrees Celsius to about 35 degrees Celsius, alternatively from about −40 degrees Celsius to about 30 degrees Celsius, alternatively from about −35 degrees Celsius to about 25 degrees Celsius, and alternatively from about −25 degrees Celsius to about 25 degrees Celsius. The low melting point oil useful herein can be chosen from vegetable oils, sucrose polyesters, alkenyl esters, hydrocarbon oils, pentaerythritol ester oils, trimethylol ester oils, citrate ester oils, glyceryl ester oils, poly alpha-olefin oils, metathesized oligomer oils, polyoils, and mixtures thereof.

The one or more oils may comprise:

### Vegetable Oils

The one or more oils may comprise one or more vegetable oils which can be liquid at room temperature. In an embodiment, acceptable vegetable oils are those with a melting point not exceeding 85 degrees Celsius. Exemplary vegetable oils can include palm oil, soybean oil, rapeseed oil, sunflower oil, peanut oil, cottonseed oil, palm kernel oil, coconut oil, olive oil, algae extract, borage seed oil, carrageenan extract, castor oil, corn oil, evening primrose oil, grape seed oil, jojoba oil, kukui nut oil, lecithin, macadamian oil, oat kernel meal oil, pea extract oil, pecan oil, safflower oil, sesame oil, shea butter, soybean oil, sunflower oil, hazelnut oil, linseed oil, rice bran oil, canola oil, flaxseed oil, walnut oil, almond oil, cocoa butter, and/or sweet almond oil.

### Sucrose Polyesters

The one or more oils may comprise one or more sucrose polyesters. Sucrose polyesters are polyester materials having multiple substitution positions around the sucrose backbone coupled with the chain length, saturation, and derivation variables of the fatty chains. Such sucrose polyesters can have an esterification (“IBAR”) of greater than about 5. In an embodiment, the one or more sucrose polyesters may have an IBAR of from about 5 to about 8, alternatively from about 5 to about 7, alternatively about 6, and alternatively about 8. As sucrose polyesters are derived from a natural resource, a distribution in the IBAR and chain length may exist. For example a sucrose polyester having an IBAR of 6, may contain a mixture of mostly IBAR of about 6, with some IBAR of about 5 and some IBAR of about 7. Additionally, such sucrose polyesters may have a saturation or iodine value (“IV”) from about 3 to about 140, alternatively from about 10 to about 120, alternatively from about 20 to about 100. Further, such sucrose polyesters can have a chain length from about C12 to about C20. Non-limiting examples of sucrose polyesters suitable for use include SEFOSE® 1618S, SEFOSE® 1618U, SEFOSE® 1618H, Sefa Soyate IMF 40, Sefa Soyate LP426, SEFOSE® 2275, SEFOSE® C1695, SEFOSE® C18:0 95, SEFOSE® C1495, SEFOSE® 1618H B6, SEFOSE® 1618S B6, SEFOSE® 1618U B6, Sefa Cottonate, SEFOSE® C1295, Sefa C895, Sefa C1095, SEFOSE® 1618S B4.5, all available from The Procter and Gamble Co. of Cincinnati, Ohio.

### Alkenyl Esters:

The one or more oils may include one or more alkenyl esters. Non-limiting examples of alkenyl esters can include oleyl myristate, oleyl stearate, oleyl oleate, and combinations thereof.

### Hydrocarbon Oils:

The one or more oils may include one or more hydrocarbon oils. Non-limiting examples of hydrocarbon oils include differing grades and molecular weights of mineral oil, liquid isoparaffin, polyisobutene, and petrolatum.

### Pentaerythritol Ester Oils and Trimethylol Ester Oils:

The one or more oils may include one or more pentaerythritol ester oils and/or one or more trimethylol ester oils. Non-lmiting examples of pentaerythritol ester oils and trimethylol ester oils can include pentaerythritol tetraisostearate, pentaerythritol tetraoleate, trimethylolpropane triisostearate, trimethylolpropane trioleate, and mixtures thereof. Such compounds are available from Kokyo Alcohol with tradenames KAKPTI, KAKTTI, and from Shin-nihon Rika with tradenames PTO and ENUJERUBU TP3SO.

### Citrate Ester Oils:

The one or more oils may include one or more citrate ester oils. Non-limiting examples of citrate ester oils can include triisocetyl citrate with tradename CITMOL 316 available from Bernel, triisostearyl citrate with tradename PELEMOL TISC available from Phoenix, and trioctyldodecyl citrate with tradename CITMOL 320 available from Bernel.

### Glyceryl Ester Oils:

The one or more oils may include one or more glyceryl ester oils. Non-limiting examples of glyceryl ester oils can include triisostearin with tradename SUN ESPOL G-318 available from Taiyo Kagaku, triolein with tradename CITHROL GTO available from Croda Surfactants Ltd., trilinolein with tradename EFADERMA-F available from Vevy, or tradename EFA-GLYCERIDES from Brooks.

### Poly Alpha-Olefin Oils:

The one or more oils may include one or more poly alpha-olefin oils. Non-limiting examples of poly α-olefin oils can include polydecenes with tradenames PURESYN 6 having a number average molecular weight of about 500, PURESYN 100 having a number average molecular weight of about 3000, and PURESYN 300 having a number average molecular weight of about 6000, all available from Exxon Mobil Co.

### Metathesized Oligomer Oils:

The one or more oils may include one or more metathesized oligomer oils derived from metathesis of unsaturated. polyol esters in amounts by weight of the composition ranging from about 0.01% to about 5%, alternatively from about 0.1% to about 1%, and alternatively from about 0.25% to about 5%. Exemplary metathesized unsaturated polyol esters and their starting materials are set forth in U.S. Patent Application U.S. 2009/0220443 A1, which is incorporated herein by reference.

A metathesized unsaturated polyol ester refers to the product obtained when one or more unsaturated polyol ester ingredient(s) are subjected to a metathesis reaction. Metathesis is a catalytic reaction that involves the interchange of alkylidene units among compounds containing one or more double bonds olefinic compounds) via the formation and cleavage of the carbon-carbon double bonds. Metathesis may occur between two of the same molecules (often referred to as self-metathesis) and/or it may occur between two different molecules (often referred to as cross-metathesis). Self-metathesis may be represented schematically as shown in Equation I:

R¹C═CHR+R²CH═CHR² [symbol] R²C═CHR¹+R²CH═CHR² (I)

where R¹ and R² are organic groups.
Cross-metathesis may be represented schematically as shown in Equation

R¹—CH═CH—R³+R¹—CH═CH—R³↔R¹—CH═CH—R³+R—CH═CH—R⁴+R²—CH═CH—R³+R²—CH═CH—R⁴+R¹—CH═CH—R¹+R²—CH═CH—R²+R³—CH═CH—R³+R⁴—CH═CH—R⁴ (II)

where R¹, R², R³, and R⁴ are organic groups.

When the unsaturated poyol ester comprises molecules that have more than one carbon-carbon double bond (i.e., a polyunsaturated polyol ester), self-metathesis results in oligomerization of the unsaturated polyol ester. The self-metathesis reaction results in the formation of metathesis diners, metathesis trimers, and metathesis tetramers. Higher order metathesis oligomers, such as metathesis pentamers and metathesis hexamers, may also be formed by continued self-metathesis and will depend on the number and type of chains connecting the unsaturated polyol ester material as well as the number of esters and orientation of the ester relative to the unsaturation.
As a starting material, metathesized unsaturated polyol esters are prepared from one or more unsaturated polyol esters. As used herein, the term “unsaturated polyol ester” refers to a compound having two or more hydroxyl groups wherein at least one of the hydroxyl groups is in the form of an ester and wherein the ester has an organic group including at least one carbon-carbon double bond. In many embodiments, the unsaturated polyol ester can he represented by the general structure I:

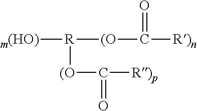

where n>l; m >0; p>0; (n+m+p)>2; R is an organic group; R is an organic group having at least one carbon-carbon double bond; and R is a saturated organic group. Exemplary embodiments of the unsaturated polyol ester are described in detail in U.S. 2009/0220443 A1.

In an embodiment, the unsaturated polyol ester is an unsaturated ester of glycerol. Sources of unsaturated polyol esters of glycerol include synthesized oils, natural oils (e.g., vegetable oils, algae oils, bacterial derived oils, and animal fats), combinations of these, and the like. Recycled used vegetable oils may also be used. Representative examples of vegetable oils include argan oil, canola oil, rapeseed oil, coconut oil, corn oil, cottonseed oil, olive oil, palm oil, peanut oil, safflower oil, sesame oil, soy-bean oil, sunflower oil, high oleoyl soy-bean oil, high oleoyl sunflower oil, linseed oil, palm kernel oil, lung oil, castor oil, high oloeyl sunflower oil, high oleoyl soybean oil, high erucic rape oils, Jatrophan oil, combinations of theses, and the like. Representative examples of animal fats include lard, tallow, chicken fat, yellow grease, fish oil, combinations of these, and the like. A representative example of a synthesized oil includes tall oil, which is a byproduct of wood pulp manufacture.

Other examples of unsaturated polyol esters can include diesters such as those derived from ethylene glycol or propylene glycol, esters such as those derived from pentaerythritol or dipentaerythritol, or sugar esters such as SEFOSE®. Sugar esters such as SEFOSE® include one or more types of sucrose polyesters as described herein, with up to eight ester groups that could undergo a metathesis exchange reaction. Other examples of suitable natural polyol esters may include but not be limited to sorbitol esters, maltitol esters, sorbitan esters, maltodextrin derived esters, xylitol esters, and other sugar derived esters.

In an embodiment, chain lengths of esters are not restricted to C8-C22 or even chain lengths only and can include natural esters that come from co-metathesis of fats and oils with short chain olefins both natural and synthetic providing a polyol ester feedstock which can have even and odd chains as well as shorter and longer chains for f metathesis reaction. Suitable short chain olefins include ethylene and butene.

The oligomers derived from the metathesis of unsaturated polyol esters may be further modified via hydrogenation. For example, in an embodiment, the oligomer can be about 60% hydrogenated or more; in certain embodiments, about 70% hydrogenated or more; in certain embodiments, about 80% hydrogenated or more; in certain embodiments, about 85% hydrogenated or more; in certain embodiments, about 90% hydrogenated or more; and in certain embodiments, generally 100% hydrogenated.

In some embodiments, the triglyceride oligomer is derived from the self-metathesis of soybean oil. The soy oligomer can include hydrogenated soy polyglycerides. The soy oligomer may also include C15-C23 alkanes, as a byproduct.An example of metathesis derived soy oligomers is the fully hydrogenated DOW CORNING® HY-3050 soy wax, available from Dow Corning.
In other embodiments, the inetathesized unsaturated polyol esters can be used as a blend with one or more non-metathesized unsaturated polyol esters. The non-metathesized unsaturated polyol esters can be fully or partially hydrogenated. Such an example is DOW CORNING® HY-3051, a blend of HY-3050 oligomer and hydrogenated soybean oil (HSBO), available from Dow Corning. In some embodiments of the invention, the non-metathesized unsaturated polyol ester is an unsaturated ester of glycerol. Sources of unsaturated polyol esters of glycerol include synthesized oils, natural oils (e.g., vegetable oils, algae oils, bacterial derived oils, and animal fats), combinations of theses, and the like. Recycled used vegetable oils may also be used. Representative examples of vegetable oils include those listed above.

Other modifications of the polyol ester oligomers can be partial amidation of some fraction of the esters with ammonia or higher organic amines such as dodecyl amine or other fatty amines. This modification will alter the overall oligomer composition but can be useful in some applications providing increased lubricity of the product. Another modification can be via partial amidation of a poly amine providing potential for some pseudo cationic nature to the polyol ester oligomers. Such an example is DOW CORNING® material HY-3200. Other exemplary embodiments of amido functionalized oligomers are described in detail in WO2012006324A1, which is incorporated herein by reference.

The polyol ester oligomers may be modified further by partial hydroformylation of the unsaturated functionality to provide one or more OH groups and an increase in the oligomer hydrophilicity.

In an embodiment, the unsaturated polyol esters and blends can be modified prior to oligomerization to incorporate near terminal branching. Exemplary polyol esters modified prior to oligomerization to incorporate terminal branching are set forth in WO2012/009525 A2, which is incorporated herein by reference.

C. Nonionic Emulsifiers

The concentrated conditioner composition may comprise from about 3° l% to about 20%, alternatively from about 5% to about 15%, and alternatively from about 7.5% to about 12% of a nonionic emulsifier, by weight of the concentrated conditioner composition. Nonionic emulsifiers may be broadly defined as including compounds containing an alkylene oxide groups (hydrophilic in nature) with a hydrophobic compound, which may be aliphatic or alkyl aromatic in nature. Examples of nonionic emulsifiers include:

1. Alcohol ethoxylates which are condensation products of aliphatic alcohols having from about 8 to about 18 carbon atoms, in either straight chain or branched chain configuration, with from about 2 to about 35 moles of ethylene oxide, e.g., a coconut alcohol ethylene oxide condensate having from about 2 to about 30 moles of ethylene oxide per mole of coconut alcohol, the coconut alcohol fraction having from about 10 to about 14 carbon atom.

2. The polyethylene oxide condensates of alkyl phenols, e.g., the condensation products of the alkyl phenols having an alkyl group containing from about 6 to about 20 carbon atoms in either a. straight chain or branched chain configuration, with ethylene oxide, the said ethylene oxide being present in amounts equal to from about 3 to about 60 moles of ethylene oxide per mole of alkyl phenol.

3. Those derived from the condensation of ethylene oxide with the product resulting from the reaction of propylene oxide and ethylene diamine products.

4. Long chain tertiary amine oxides such as those corresponding to the following general formula: R1R2R3N→O wherein R1 contains an alkyl, alkenyl or monohydroxy alkyl redical of from about 8 to about 18 carbon atoms, from 0 to about 10 ethylene oxide moieties, and from 0 to about 1 glyceryl moiety, and R2 and R3 contain from about 1 to about 3 carbon atoms and from 0 to about 1 hydroxy group, e.g., methyl, ethyl, propyl, hydroxyethyl, or hydroxypropyl radicals (the arrow in the formula represents a semipolar bond).

5. Long chain tertiary phosphine oxides corresponding to the following general formula: RR′R″P→O wherein R contains an alkyl, alkenyl or monohydroxyalkyl radical ranging from about 8 to about 18 carbon atoms in chain length, from 0 to about 10 ethylene oxide moieties and from 0 to about 1 glyceryl moiety and R′ and R″ are each alkyl or monohydroxyalkyl groups containing from about 1 to about 3 carbon atoms. The arrow in the formula represents a semipolar bond.

6. Long chain dialkyl sulfoxides containing one short chain alkyl or hydroxy alkyl radical of from about 1 to about 3 carbon atoms (usually methyl) and one long hydrophobic chain which include alkyl, alkenyl, hydroxy alkyl, or keto alkyl radicals containing from about 8 to about 20 carbon atoms, from 0 to about 10 ethylene oxide moieties and from 0 to about 1 glyceryl moiety.

7. Polysorbates, e.g., sucrose esters of fatty acids. Such materials are described in U.S. Pat. No. 3,480,616, e.g., sucrose cocoate (a mixture of sucrose esters of a coconut acid, consisting primarily of monoesters, and sold under the tradenames GRILLOTEN LSE 87K from RITA, and CRODESTA SL-40 from Croda).

8. Alkyl polysaccharide nonionic emulsifiers are disclosed in U.S. Pat. No. 4,565,647, Llenado, issued Jan. 21, 1986, having a hydrophobic group containing from about 6 to about 30 carbon atoms, preferably from about 10 to about 16 carbon atoms and a polysaccharide, e.g., a polyglycoside, hydrophilic group. The polysaccharide can contain from about 1.0 to about 10, alternatively from about 1.3 to about 3, and alternatively from about 1.3 to about 2.7 saccharide .s. Any reducing saccharide containing 5 or 6 carbon atoms can be used, e.g., glucose, galactose and galactosyl moieties can be substituted for the glucosyl moieties. (Optionally the hydrophobic group is attached at the 2-, 3-, 4-, etc. positions thus giving a glucose or galactose as opposed to a glucoside or galactoside.) The intersaccharide bonds can be, e.g., between the one position of the additional saccharide units and the 2-, 3-, 4-, and/or 6-positions on the preceding saccharide units. Optionally there can be a polyalkyleneoxide chain joining the hydrophobic moiety and the polysaccharide moiety. The alkyl group preferably contains up to about 3 hydroxy groups and/or the polyalkyleneoxide chain can contain up to about 10, preferably less than 5, alkylene moieties. Suitable alkyl polysaccharides are octyl., nonyldecyl, undecyldodecyl, tridecyl, tetradecyl, pentadecyl, hexadecyl, heptadecyl, and octadecyl, di-, tri-, tetra-, penta-, and hexaglucosides, galactosides, lactosides, glucoses, fructosides, fructoses and/or galactoses.

9. Polyethylene glycol (PEG) glyceryl fatty esters, as depicted by the formula I. C(O)OCH₂ CH(OH)CH₂ (OCH₂ CH₂)n OH wherein i is from about 5 to about 200, preferably from about 20 to about 100, more preferably from about 30 to about 85, and RC(O)— is an ester wherein R comprises an aliphatic radical having from about 7 to 19 carbon atoms, preferably from about 9 to 17 carbon atoms, more preferably from about 11 to 17 carbon atoms, most preferably from about 11 to 14 carbon atoms. In an embodiment, the combinations of n may be from about 20 to about 100, with C12-C18, alternatively C12-C15 fatty esters, for minimized adverse effect on foaming.

In an embodiment, the nonionic emulsifier may be a silicone emulsifier. A wide variety of silicone emulsifiers may be useful herein. These silicone emulsifiers are typically organically modified siloxanes, also known to those skilled in the art as silicone surfactants. Useful silicone emulsifiers include dimethicone copolyols. These materials are polydimethyl siloxanes which have been modified to include polyether side chains such as polyethylene oxide chains, polypropylene oxide chains, mixtures of these chains, and polyether chains containing moieties derived from both ethylene oxide and propylene oxide. Other examples include alkyl-modified dimethicone copolyols, i.e., compounds which contain C2-C30 pendant side chains. Still other useful dimethicone copolyols include materials having various cationic, anionic, amphoteric, and zwitterionic pendant moieties.

In an embodiment, the nonionic emulsifier may have a hydrocarbon chain length of from about 16 to about 20 carbon atoms and from about 20 to about 25 moles of ethoxylate.

In an embodiment, the nonionic emulsifier may have a hydrocarbon chain length of from about 19 to about 11, alternatively from about 9 to about 11 carbon atoms, and from about 2 to about 4 moles of ethoxylate.

In an embodiment, the nonionic emulsifier may comprise a combination of (a) a nonionic emulsifier having a hydrocarbon chain that is branched, has a length of from about 11 to about 15 carbon atoms, and has from about 5 to about 9 moles of ethoxylate; and (b) a nonionic emulsifier having a hydrocarbon chain that has a length of from about 11 to about 13 carbon atoms and has from about 9 to about 12 moles of ethoxylate.

The nanoemulsions used in this invention may be prepared by two different methods: (1) mechanical, and (2) emulsion polymerization.

The first method of preparing the nanoemulsion is the mechanical method in which the nanoemulsion is prepared via the following steps: (1) a primary surfactant is dissolved in water, (2) a silicone is added, and a two-phase mixture is formed, (3) with simple mixing, a co-surfactant is slowly added to the two-phase mixture, until a clear isotropic microemulsion of a siloxane-in-water is formed.

The second method of preparing the nanoemulsion is by emulsion polymerization. Emulsion polymerization methods for making nanoemulsions of polymers involve starting with polymer precursors, i.e., monomers, or reactive oligomers, which are immiscible in water; a surfactant to stabilize polymer precursor droplets in water; and a water soluble polymerization catalyst. Typically, the catalyst is a strong mineral acid such as hydrochloric acid, or a strong alkaline catalyst such as sodium hydroxide. These components are added to water, the mixture is stirred, and polymerization is allowed to advance until the reaction is complete, or the desired degree of polymerization (DP) is reached, and an emulsion of the polymer is formed.

The oils may be selected from the group consisting of organic conditioning material such as oil or wax, either alone or in combination with other conditioning agents, such as the silicones described above. The organic material can be non-polymeric, oligomeric or polymeric. It may be in the form of oil or wax and may be added in the formulation neat or in a pre-emulsified form. Some non-limiting examples of organic conditioning materials include, but are not limited to: i) hydrocarbon oils; ii) polyolefins.

D. Perfume

The concentrated conditioner composition may comprise from about 0.5% to about 7%, alternatively from about 1% to about 6%, and alternatively from about 2% to about 5% perfume, by weight of the concentrated conditioner composition. In an embodiment, the concentrated conditioner composition may comprise from about 0.5% to about 4%, alternatively from about 0.75% to about 3%, and alternatively from about 1% to about 2.5% perfume, by weight of the concentrated conditioner composition.

In an embodiment, the concentrated conditioner composition may have a silicone to perfume ratio of from about 98:2 to about 50:50, alternatively from about 95:5 to about 50:50, alternatively from about 90:10 to about 60:40, and alternatively from about 85:15 to about 70:30.

Examples of suitable perfumes may be provided in the CTFA (Cosmetic, Toiletry and Fragrance Association) 1992 International Buyers Guide, published by CFTA Publications and OPD 1993 Chemicals Buyers Directory 80th Annual Edition, published by Schnell Publishing Co. A plurality of perfume components may be present in the concentrated conditioner composition.

E. Conditioner High Melting Point Fatty Compounds

The concentrated conditioner composition may comprise less than 10% conditioner high melting point fatty compounds, alternatively less than 8% conditioner high melting point fatty compounds, alternatively less than 6% conditioner high melting point fatty compounds, alternatively may be substantially free of conditioner high melting point fatty compounds, and alternatively may comprise 0% conditioner high melting point fatty compounds, by weight of the concentrated conditioner composition. In one embodiment, the concentrated conditioner composition may comprise less than 5% conditioner high melting point fatty compounds, alternativey less than 4% conditioner high melting point fatty compounds, alternatively less than 3% conditioner high melting point fatty compounds, alternatively less than 2% conditioner high melting point fatty compounds, alternatively less than 1% conditioner high melting point fatty compounds, and alternatively may comprise 0% conditioner high melting point fatty compounds. The concentrated conditioner composition may have a silicone to conditioner high melting point fatty compounds ratio of from about 100:0 to about 40:60, alternatively from about 100:0 to about 50:50, and alternatively from about 100:0 to about 60:40, alternatively from about 100:0 to about 70:30.

The high melting point fatty compounds have a melting point of about 25° C. or higher, and are selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than about 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

The fatty alcohols described herein are those having from about 14 to about 30 carbon atoms, preferably from about 16 to about 22 carbon atoms. These fatty alcohols are saturated and can be straight or branched chain alcohols. Nonlimiting examples of fatty alcohols include cetyl alcohol, stearyl alcohol, behenyl alcohol, and mixtures thereof.

The fatty acids useful herein are those having from about 10 to about 30 carbon atoms, preferably from about 12 to about 22 carbon atoms, and more preferably from about 16 to about 22 carbon atoms. These fatty acids are saturated and can be straight or branched chain acids. Also included are diacids, triacids, and other multiple acids which meet the requirements herein. Also included herein are salts of these fatty acids. Nonlimiting examples of fatty acids include lauric acid, palmitic acid, stearic acid, behenic acid, sebacic acid, and mixtures thereof.

The fatty alcohol derivatives and fatty acid derivatives useful herein include alkyl ethers of fatty alcohols, alkoxylated fatty alcohols, alkyl ethers of alkoxylated fatty alcohols, esters of fatty alcohols, fatty acid esters of compounds having esterifiable hydroxy groups, hydroxy-substituted fatty acids, and mixtures thereof. Nonlimiting examples of fatty alcohol derivatives and fatty acid derivatives include materials such as methyl stearyl ether; the ceteth series of compounds such as ceteth-1 through ceteth-45, which are ethylene glycol ethers of cetyl alcohol, wherein the numeric designation indicates the number of ethylene glycol moieties present; the steareth series of compounds such as steareth-1 through steareth-10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e., a mixture of fatty alcohols containing predominantly cetyl and stearyl alcohol, wherein the numeric designation indicates the number of ethylene glycol moieties present; C16-C30 alkyl ethers of the ceteth, steareth, and ceteareth compounds just described; polyoxyethylene ethers of behenyl alcohol; ethyl stearate, cetyl stearate, cetyl palmitate, stearyl stearate, myristyl myristate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, glyceryl monostearate, glyceryl distearate, glyceryl tristearate, and mixtures thereof.

In an embodiment, the fatty compound may be a single high melting point compound of high purity. Single compounds of pure fatty alcohols selected may be selected from the group consisting of pure cetyl alcohol, stearyl alcohol, and behenyl alcohol. By “pure” herein, what is meant is that the compound has a purity of at least about 90%, alternatively at least about 95%.

Commercially available conditioner high melting point fatty compounds described herein include: cetyl alcohol, stearyl alcohol, and behenyl alcohol having tradenames KONOL series available from Shin Nihon Rika (Osaka, Japan), and NAA series available from NOF (Tokyo, Japan); pure behenyl alcohol having tradename 1-DOCOSANOL available from WAKO (Osaka, Japan), various fatty acids having tradenames NEO-FAT available from Akzo (Chicago, Ill. USA), HYSTRENE available from Witco Corp. (Dublin, Ohio USA), and DERMA available from Vevy (Genova, Italy).

F. Cationic Surfactants

In an embodiment, the concentrated conditioner composition may comprise 0%, alternatively from about 0.25% to about 5%, alternatively from about 0.5% to about 4%, and alternatively from about 1% to about 3% cationic surfactants, by weight of the concentrated conditioner composition.

The cationic surfactant may be a mono-long alkyl quaternized ammonium salt having the formula (XIII) [from WO2013148778]:

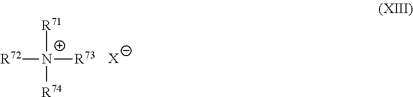

wherein one of R⁷¹, R⁷²R⁷³ a n R⁷⁴ selected from an aliphatic group of from about 14 to about 30 carbon atoms or an aromatic, alkoxy, polyoxyalkylene, alkylamido, hydroxyalkyl, aryl or alkylaryl group having up to about 30 carbon atoms; the remainder of R⁷¹, R⁷²R⁷³ and R⁷⁴ are independently selected from an aliphatic group of from about 1 to about 8 carbon atoms or an aromatic, alkoxy, polyoxyalkylene, alkylamido, hydroxyalkyl, aryl or alkylaryl group having up to about 8 carbon atoms; and X is a salt-forming anion such as those selected from halogen, (e.g., chloride, bromide), acetate, citrate, lactate, glycolate, phosphate, nitrate, sulfonate, sulfate, alkylsulfate, glutamate, and alkyl sulfonate radicals. The aliphatic groups can contain, in addition to carbon and hydrogen atoms, ether linkages, and other groups such as amino groups. The longer chain aliphatic groups, e.g., those of about 16 carbons, or higher, can be saturated or unsaturated. Preferably, one of R⁷¹ , R⁷² R⁷³ and R⁷⁴ is selected from an alkyl group of from about 14 to about 30 carbon atoms, more preferably from about 16 to about 22 carbon atoms, still more preferably from about 16 to about 18 carbon atoms; the remainder of R⁷¹, R⁷² , R⁷³, and R⁷⁴ are independently selected from the group consisting of CH_{3,} C₂H_{5,} C₂H₄OH, CH₂C₅H₅, and mixtures thereof; and (X) is selected from the group consisting of Cl, Br, CH₃OSO₃, and mixtures thereof. It is believed that such mono-long alkyl quatemized ammonium salts can provide improved slippery and slick feel on wet hair.

Nonlimiting examples of such mono-long alkyl quatemized ammonium salt cationic surfactants include: behenyl trimethyl ammonium chloride available, for example, with tradename Genamine KDMP from Clariant, with tradename INCROQUAT TMC-80 from Croda and ECONOL TM22 from Sanyo Kasei; stearyl trimethyl ammonium chloride available, for example, with tradename CA-2450 from Nikko Chemicals; cetyl trimethyl ammonium chloride available, for example, with tradename CA-2350 from Nikko Chemicals; behenyltrimethylammonium methyl sulfate, available from FeiXiang; hydrogenated tallow alkyl trimethyl ammonium chloride; stearyl dimethyl benzyl ammonium chloride; and stearoyl amidopropyl dimethyl benzyl ammonium chloride.

Among them, more preferred cationic surfactants are those having a shorter alkyl group, i.e., C₁₆ alkyl group. Such cationic surfactant includes, for example, cetyl trimethyl ammonim chloride. It is believed that cationic surfactants having a shorter alkyl group are advantageous for concentrated hair care silicone nanoemulsion compositions of the present invention comprising a cationic surfactant and with improved shelf stability.

G. Water Miscible Solvents

The concentrated conditioner compositions described herein may comprise from about 0.1% to about 25%, alternatively from about 0.1% to about 20%, and alternatively from about 0.1% to about 15% of a water miscible solvent, by weight of the concentrated conditioner composition. Non-limiting examples of suitable water miscible solvents include polyols, copolyols, polycarboxylic acids, polyesters and alcohols.

Examples of useful polyols include, but are not limited to, glycerin, diglycerin, propylene glycol, ethylene glycol, butylene glycol, pentylene glycol, 1,3-butylene glycol, cyclohexane dimethanol, hexane diol, polyethylene glycol (200-600), sugar alcohols such as sorbitol, manitol, lactitol and other mono- and polyhydric low molecular weight alcohols (e.g., C₂-C₈ alcohols); mono di- and oligo-saccharides such as fructose, glucose, sucrose, maltose, lactose, and high fructose corn syrup solids and ascorbic acid.

Examples of polycarboxylic acids include, but are not limited to citric acid, maleic acid, succinic acid, polyacrylic acid, and polymaleic acid.

Examples of suitable polyesters include, but are not limited to, glycerol triacetate, acetylated-monoglyceride, diethyl phthalate, triethyl citrate, tributyl citrate, acetyl triethyl citrate, acetyl tributyl citrate.

Examples of suitable dimethicone copolyols include, but are not limited to, PEG-12 dimethicone, PEG/PPG-18/18 dimethicone, and PPG-12 dimethicone.

Examples of suitable alcohols include, but are not limited to ethanol, n-propanol, isopropanol, n-butanol, sec-butanol, tert-butanol, n-hexanol and cyclohexanol.

Other suitable water miscible solvents include, but are not limited to, alkyl and allyl phthalates; napthalates; lactates (e.g., sodium, ammonium and potassium salts); sorbeth-30; urea; lactic acid; sodium pyrrolidone carboxylic acid (PCA); sodium hyraluronate or hyaluronic acid; soluble collagen; modified protein; monosodium L-glutamate; alpha & beta hydroxyl acids such as glycolic acid, lactic acid, citric acid, maleic acid and salicylic acid; glyceryl polymethacrylate; polymeric plasticizers such as polyquaterniums; proteins and amino acids such as glutamic acid, aspartic acid, and lysine; hydrogen starch hydrolysates; other low molecular weight esters (e.g., esters of C₂-C₁₀ alcohols and acids); and any other water soluble plasticizer known to one skilled in the art of the foods and plastics industries; and mixtures thereof.

In an embodiment, the water miscible solvents may be selected from the group consisting of glycerin, propylene glycol, dipropylene glycol, and mixtures thereof. EP 0283165 B1 discloses other suitable water miscible solvents, including glycerol derivatives such as propoxylated glycerol.

H. Viscosity Modifiers

The concentrated conditioner composition described herein may comprise from about 0.1% to about 2%, alternatively from about 0.1% to about 1%, and alternatively from about 0.1% to about 0.5% of a viscosity modifier, by weight of the concentrated conditioner composition. Non-limiting examples of suitable viscosity modifiers include water soluble polymers, cationic water soluble polymers,

Examples of water soluble polymers include, but are not limited to (1) vegetable based polymers such as gum Arabic, tragacanth gum, galactan, guar gum, carob gum, karaya gum, carrageenan, pectin, agar, quince seed, algal colloid, starch (rice, corn, potato, or wheat), and glycyrrhizinic acid; (2) microorganism-based polymers such as xanthan gum, dextran, succinoglucan, and pullulan; and (3) animal-based polymers such as collagen, casein, albumin, and gelatin. Examples of semi-synthetic water-soluble polymers include (1) starch-based polymers such as carboxymethyl starch and methylhydroxypropyl starch; (2) cellulose-based polymers such as methylcellulose, nitrocellulose, ethylcellulose, methylhydroxypropylcellulose, hydroxyethylcellulose, sodium cellulose sulfate, hydroxypropylcellulose, sodium carboxymethylcellulose (CMC), crystalline cellulose, and cellulose powder; and (3) alginate-based polymers such as sodium alginate and propylene glycol alginate. Examples of synthetic water-soluble polymers include (1) vinyl-based polymers such as polyvinyl alcohol, polyvinyl methyl ether-based polymer, polyvinylpyrrolidone, and carboxyvinyl polymer (CARBOPOL 940, CARBOPOL 941; (2) polyoxyethylene-based polymers such as polyethylene glycol 20,000, polyethylene glycol 6,000, and polyethylene glycol 4,000; (3) copolymer-based polymers such as a copolymer of polyoxyethylene and polyoxypropylene, and PEG/PPG methyl ether; (4) acryl-based polymers such as poly(sodium acrylate), poly(ethyl acrylate), polyacrylamide, polyethylene imines, and cationic polymers. The water-swellable clay minerals are nonionic water-soluble polymers and correspond to one type of colloid-containing aluminum silicate having a triple layer structure. More particular, as examples thereof, mention may be made of bentonite, montmorillonite, beidellite, nontronite, saponite, hectorite, aluminum magnesium silicate, and silicic anhydride.

Examples of cationic water soluble polymers include, but are not limited to (1) quaternary nitrogen-modified polysaccharides such as cation-modified cellulose, cation-modified hydroxyethylcellulose, cation-modified guar gum, cation-modified locust bean gum, and cation-modified starch; (2) dimethyldiallylammonium chloride derivatives such as a copolymer of dimethyldiallylammonium chloride and acrylamide, and poly(dimethylmethylene piperidinium chloride); (3) vinylpyrrolidone derivatives such as a salt of a copolymer of vinylpyrrolidone and dimethylaminoethyl methacrylic acid, a copolymer of vinylpyrrolidone and methacrylamide propyltrimethylammonium chloride, and a copolymer of vinylpyrrolidone and methylvinylimidazolium chloride; and (4) methacrylic acid derivatives such as a copolymer of methacryloylethyldimethylbetaine, methacryloylethyl trimethylammonium chloride and 2-hydroxyethyl methacrylate, a copolymer of methacryloylethyldimethylbetaine, and methacryloylethyl trimethylammonium chloride and methoxy polyethylene glycol methacrylate.

I. Viscosity

The concentrated conditioner composition described herein may have a liquid phase viscosity of from about 1 centipoise to about 3,000 centipoise, alternatively from about 1 centipoise to about 2,500 centipoise, alternatively from about 5 centipoise to about 2,000 centipoise, alternatively from about 10 centipoise to about 1,500 centipoise, and alternatively from about 15 centipoise to about 1,000 centipoise. In an embodiment, the concentrated conditioner composition described herein may have a liquid phase viscosity of from about 1 centipoise to about 10,000 centipoise, alternatively from about 1 centipoise to about 7,500 centipoise, alternatively from about 5 centipoise to about 5,000 centipoise, alternatively from about 10 centipoise to about 2,500 centipoise, and alternatively from about 15 centipoise to about 1,000 centipoise. In an embodiment, the concentrated conditioner composition described herein may have a liquid phase viscosity of from about 500 centipoise to about 15,000 centipoise, alternatively from about 1,000 centipoise to about 12,500 centipoise, alternatively from about 1,500 centipoise to about 10,000 centipoise, alternatively from about 2,000 centipoise to about 7,500 centipoise, and alternatively from about 2,500 centipoise to about 5,000 centipoise The concentrated hair composition viscosity values may be measured using a TA Instruments AR-G2 Rheometer with a concentric cylinder attachment at a shear rate of 100 reciprocal seconds at 25° C.

### Optional Ingredients

The shampoo composition and the concentrated conditioner composition described herein may optionally comprise one or more additional components known for use in hair care or personal care products, provided that the additional components are physically and chemically compatible with the essential components described herein, or do not otherwise unduly impair product stability, aesthetics or performance. Such optional ingredients are most typically those materials approved for use in cosmetics and that are described in reference books such as the CTFA Cosmetic Ingredient Handbook, Second Edition, The Cosmetic, Toiletries, and Fragrance Association, Inc. 1988, 1992. Individual concentrations of such additional components may range from about 0.001 wt % to about 10 wt % by weight of the conditioning composition.

Emulsifiers suitable as an optional ingredient herein include mono- and di-glycerides, fatty alcohols, polyglycerol esters, propylene glycol esters, sorbitan esters and other emulsifiers known or otherwise commonly used to stabilized air interfaces, as for example those used during preparation of aerated foodstuffs such as cakes and other baked goods and confectionary products, or the stabilization of cosmetics such as hair mousses.

Further non-limiting examples of such optional ingredients include preservatives, perfumes or fragrances, cationic polymers, viscosity modifiers, coloring agents or dyes, conditioning agents, hair bleaching agents, thickeners, moisturizers, foam boosters, additional surfactants or nonionic cosurfactants, emollients, pharmaceutical actives, vitamins or nutrients, sunscreens, deodorants, sensates, plant extracts, nutrients, astringents, cosmetic particles, absorbent particles, adhesive particles, hair fixatives, fibers, reactive agents, skin lightening agents, skin tanning agents, anti-dandruff agents, perfumes, exfoliating agents, acids, bases, humectants, enzymes, suspending agents, pH modifiers, hair colorants, hair perming agents, pigment particles, anti-acne agents, anti-microbial agents, sunscreens, tanning agents, exfoliation particles, hair growth or restorer agents, insect repellents, shaving lotion agents, non-volatile solvents or diluents (water-soluble and water-insoluble), co-solvents or other additional solvents, and similar other materials.

In an embodiment, the optional ingredients include anti-dandruff agents which may be selected from: pyridinethione salts, azoles (e.g.,ketoconazole, econazole, and elubiol), selenium sulfide, particulate sulfur, salicylic acid, and mixtures thereof. A typical anti-dandruff agent is pyridinethione salt. Hair care compositions can also include a zinc-containing layered material. An example of a zinc-containing layered material can include zinc carbonate materials. Of these, zinc carbonate and pyridinethione salts (particularly zinc pyridinethione or “ZPT) are common in the composition, and often present together.

### Aerosol Foam Dispenser

The shampoo composition and/or the concentrated conditioner composition may be provided in an aerosol foam dispenser. The aerosol foam dispenser may comprise a reservoir for holding the concentrated conditioner composition. The reservoir may be made out of any suitable material selected from the group consisting of plastic, metal, alloy, laminate, and combinations thereof. In an embodiment, the reservoir may be for one-time use. In an embodiment, the reservoir may be removable from the aerosol foam dispenser. Alternatively, the reservoir may be integrated with the aerosol foam dispenser. In an embodiment, there may be two or more reservoirs.

In an embodiment, the reservoir may be comprised of a material selected from the group consisting of rigid materials, flexible materials, and combinations thereof. The reservoir may be comprised of a rigid material if it does not collapse under external atmospheric pressure when it is subject to an interior partial vacuum.

The hair care composition in the form of a foam, can have a density of from about 0.025 g/cm³ to about 0.30 g/cm³, alternatively from about 0.035 g/cm³ to about 0.20 g/cm³, alternatively from about 0.05 g/cm³ to about 0.15 g/cm³, and alternatively from about 0.075 g/cm³ to about 0.12 g/cm³. In an embodiment, the foam has a density of from about 0.025 g/cm³ to about 0.15 g/cm³, alternatively from about 0.05 g/cm³ to about 0.12 g/cm³, and alternatively from about 0.075 g/cm³ to about 0.10 g/cm³.

The concentrated compositions of the present invention are dispensed at a dosage of from about 1 grams to about 6 grams, alternatively from about 2 grams to about 6 grams, alternatively from about 2 grams to about 6 grams, and alternatively from about 3 grams to about 6 grams per the intended use by the consumer.

### Propellant

The shampoo composition and/or the concentrated conditioner composition described herein may comprise from about 1% to about 10%, alternatively from about 1% to about 6% propellant, alternatively from about 2% to about 5% propellant, and alternatively from about 3% to about 4% propellant, by weight of the shampoo and/or concentrated conditioner composition.

The propellant may comprise one or more volatile materials, which in a gaseous state, may carry the other components of the concentrated conditioner composition in particulate or droplet form. The propellant may have a boiling point within the range of from about −45° C. to about 5° C. The propellant may be liquefied when packaged in convention aerosol containers under pressure. The rapid boiling of the propellant upon leaving the aerosol foam dispenser may aid in the atomization of the other components of the concentrated conditioner composition.

Aerosol propellants which may be employed in the aerosol composition , include the chemically-inert hydrocarbons such as propane, n-butane, isobutane, cyclopropane,and mixtures thereof, as well as halogenated hydrocarbons such as dichlorodifluoromethane, 1,1-dichloro-1,1,2,2-fluoroethane, 1-chloro-1,1-difluoro-2,2-trifluoroethane, 1-chloro-1,1-difluoroethylene, 1,1-difluoroethane, dimethyl ether, monochlorodifluoromethane, trans-1,3,3,3-tetrafluoropropene, and mixtures thereof. The propellant may comprise hydrocarbons such as isobutane, propane, and butane these materials may be used for their low ozone reactivity and may be used as individual components where their vapor pressures at 21.1° C. range from about 1.17 Bar to about 7.45 Bar, alternatively from about 1.17 Bar to about 4.83 Bar, and alternatively from about 2.14 Bar to about 3.79 Bar. The propellant may be hydrofluoroolefins (HFOs).

### EXAMPLES AND DATA

The following examples illustrate the shampoo compositions and concentrated conditioner compositions described herein. The exemplified compositions can be prepared by conventional formulation and mixing techniques. It will be appreciated that other modifications of the present invention within the skill of those in the shampoo formulation art can be undertaken without departing from the spirit and scope of this invention. All parts, percentages, and ratios herein are by weight unless otherwise specified. Some components may come from suppliers as dilute solutions. The amount stated reflects the weight percent of the active material, unless otherwise specified.

Three “Clarifying” shampoos are employed in the below examples that were void of high melting point fatty compounds and conditioning agents. One was a Pantene clarifying shampoo and the other two were concentrated foam shampoos. The concentrated foam shampoos may be prepared by mixing together water and surfactants along with any solids that need to be melted at an elevated temperature, e.g. about 75° C. The ingredients are mixed thoroughly at the elevated temperature and then cooled to ambient temperature. Additional ingredients, including electrolytes, polymers, silicone emulsions, preservatives and fragrances may be added to the cooled product.

TABLE 1
Concentrated Aerosol Foam Cleansing Shampoo Composition

| | Foam | Foam |
|---|---|---|
| Raw Material | Shampoo 1 | Shampoo 2 |
| Sodium Undecyl Sulfate | 23.6 | |
| (C11 70% active)¹ | | |
| Lauramrdopropyl Betaine | 5.8 | |
| (LAPB 35% active)² | | |
| Para Hydroxy Phenyl Butanone³ | 3.9 | |
| Alkyl polyglucosrde⁴ | | 25.2 |
| Cocamidopropyl betaine | | 3.9 |
| Polyvinyl alcohol⁵ | 1.9 | 1.9 |
| Perfume | 2.3 | 2.3 |
| Citric Acid | 0.3 | 0.3 |
| Preservative (Kathon) | 0.03 | 0.03 |
| Propellant (Aeron-46) | 3.1 | 3.1 |
| Water | q.s. | (q.s.) |
| Weight % of high melting point | 0% | 0% |
| fatty compounds | | |

¹Sodium Undecyl Sulfate (C11, Isachem 123S) at 70% active, supplier: P&G
²LAPB (Mackam DAB), at 35% active level, supplier: Rhodia
³Raspberry Ketone, supplier: Spectrum
⁴EcoSense 919 available from Dow Chemical.
⁵PVA-403 available from Kuraray

TABLE 2
Concentrated Liquid and Aerosol Foam Cleansing Shampoo Composition

| Ingredient | Ex. 21 | Ex. 22 | Ex 23 | Ex. 31 | Ex. 32 | Ex 33 |
|---|---|---|---|---|---|---|
| Form | Liquid | | | Aerosol foam | | |
| Bulk Viscosity (cps) | 243 | 444 | 411 | 243 | 444 | 411 |
| Water | q.s. | q.s. | q.s. | q.s. | q.s. | q.s. |
| Sodium Undecyl Sulfate (C11 | 24 | 24 | 24 | 24 | 24 | 24 |
| 70% active)¹ | | | | | | |
| Lauramidopropyl Betaine (LAPB | 6 | 6 | 6 | 6 | 6 | 6 |
| 35% active)² | | | | | | |
| Para Hydroxy Phenyl Butanone³ | 4 | 4 | 4 | 4 | 4 | 4 |
| Guar, Hydroxylpropyl | — | 0.4 | 0.4 | — | 0.4 | 0.4 |
| Trimonium Chloride, Jaguar C- | | | | | | |
| 500⁴ | | | | | | |
| Polyquatenrium⁵ | — | — | 0.2 | | | 0.2 |
| Silicone quaternium⁶ | — | — | 2 | | | 2 |
| Fragrance | 2.4 | 2.4 | 2.4 | 2.4 | 2.4 | 2.4 |
| Sodium Chloride | | Adjust as needed for viscosity | | | | |
| Preservatives, pH adjusters | | Up to 1% | | | | |
| Propellant A46⁷ | — | — | | 4 | 4 | 4 |

¹Sodium Undecyl Sulfate (C11, Isachem 123S) at 70% active, supplier: P&G
²LAPB (Mackam DAB), at 35% active level, supplier: Rhodia
³Raspberry Ketone, supplier: Spectrum
⁴Jaguar C500, MW of 500,000, CD of 0.8, from Rhodia
⁵Polydadmac, trade name: Mirapol 100s, from Rhodia
⁶Silicone quaternium micro-emulsion, 30% active, Abil ME 45, from Evonik
⁷Aeron A-Blends, A46 (Isobutane/Propane = 84.85/15.15) from Diversified CPC International

The following aerosol conditioner compositions may be prepared by weighing distilled water and the aminosilicone emulsions into a stainless steel beaker. The beaker is placed in a water bath on a hot plate while mixing with overhead mixer at 100 to 150 rpm. If fatty alcohols are present in the formula, the cetyl alcohol and stearyl alcohol are added and the mixture is heated to 70-75 C. Cetyltrimethylammonium choloride is then added and mixing speed is increased to 250-350 rpm due to viscosity increase. When the materials are all heated thoroughly and homogenous, the heating is stopped while the mixture is continued to stir. The batch is cooled to 35C by removing the hot water from the water bath and replacing with cold water. The perfume and Kathon are added and with continued stirring for ˜10 minutes. For foaming, the batch is transferred to appropriate container and propellant Aeron-46 is added.

TABLE 3
Concentrated Aerosol Foam Conditioner Composition

| Raw Material | Ex 1 | Ex 2 | Ex 3 | Ex 4 |
|---|---|---|---|---|
| Aminosilicone¹ | 12 | 12 | 12 | 12 |
| Perfume | 2.4 | 2.4 | 3.0 | 3.0 |
| Cetyltrimethylammonium Chloride | | 2.5 | 2.5 | 2.5 |
| Cetyl Alcohol | | | 1.5 | 3.0 |
| Stearyl Alcohol | | | 1.5 | 3.0 |
| Preservative (Kathon) | 0.03 | 0.03 | 0.03 | 0.03 |
| Propellant (Aeron-46) | 4.0 | 4.0 | 4.0 | 4.0 |
| Water | (q.s.) | (q.s.) | (q.s.) | (q.s.) |
| Weight ratio of oil to high melting | 100:0 | 100:0 | 80:20 | 67:33 |
| point fatty compounds | | | | |

¹Silsoft 253 (20% active) available from Momentive

TABLE 4
Concentrated liquid and Aerosol Foam Conditioner Composition

| Ingredient | Ex51 | Ex53 | Ex. 61 | Ex. 63 |
|---|---|---|---|---|
| Bulk Viscosity (cps) | <50 | 1200 | C50 | 1200 |
| Aerosol Foam collapsing time* | | | + | +++ |
| Silicone/Perfume ratio | 4 | 4 | 4 | 4 |
| Water | q.s. | q.s. | q.s. | q.s. |
| Cetrimonium chloride ¹ | − | 2.5 | − | 2.5 |
| Aminosilicone micro-emulsion ² | 12 | 12 | 12 | 12 |
| Cetyl alcohol ³ | − | 3 | − | 3 |
| Stearyl alcohol ⁴ | − | 3 | − | 3 |
| Propellant ⁵ | − | − | 6 | 6 |
| Fragrance | 3 | 3 | 3 | 3 |

| | |
|---|---|
| Preservatives, pH adjusters | Up to 1% |

*Aerosol Foam collapsing time- + means acceptable − foam stands > 1 min for not collapsing after dispensing, +++ means foam stands > 10 min for not collapsing after dispensing.
¹ CTAC (Varisoft 100), available from Evonik
² Aminosilicone micro-emulsion (Silsoft 253), available from Momentive
³ Cetyl alcohol, available from Procter & Gamble
⁴ Stearyl alcohol, available from Procter & Gamble
⁵ A46 (Isobutane/Propane = 84.8/15.2) available from Diversified CPC International

Each of the above conditioners was treated onto General Population brown hair switches as part of a regimen with Pantene Pro-V Clarifying Shampoo for up to 10 treatment cycles. As a regimen control, the Pantene Pro-V Clarifying Shampoo was combined with Pantene Anti-Breakage Conditioner. The latter is known to have an aminosilicone content of 2.5% and a total high melting point fatty compounds (cetyl and stearyl alcohols) content of 5.20% for a weight ratio of oil to high melting point fatty compounds of 32.5:67.5. Wet and dry combing data was collected at cycles 1 and 10 of the shampoo+conditioner regimen. Images were taken of the hair switches after 3 and 10 regimen treatment cycles to assess hair volume. In order to determine the durability of the conditioning, the hair switches were then subjected to shampooing alone with the Pantene Clarifying Shampoo for up to 10 cycles and with wet and dry combing data collected at shampoo only cycles 1, 2, 5 and 10. Images were taken of the hair switches after 5 and 10 shampoo only cycles to assess hair volume.

### Multiple Cycle Shampoo Plus Conditioner Treatments:

1. Six 4 gram, 8 inch General Population brown hair switches are wet with 100 degrees Fahrenheit water at a sink (bound on root-ends with glue/tape and hanging on metal holder) with a shower head fixture (flow rate is 1.5 gallons per minute) for 15 to 20 seconds.
2. Liquid shampoos are applied at 0.1grams of product per gram of hair (e.g., Pantene Pro-V Clarifying Shampoo) via a syringe and milked/scrubbed for 30 seconds followed by a 30 seconds shower head rinse (with gentle manipulation at top of switch to ensure uniform rinsing). Concentrated liquid foam shampoos are applied at 0.05 grams of product per gram of hair with a spatula (foam is dispensed in weigh boat and applied weight recorded) and following the same application procedure.
3. Liquid conditioners are applied at a 0.1 grams of product per gram of hair (e.g., Pantene Moisture Renewal Conditioner etc.) via a syringe (weighed on weigh scale) evenly over the hair switch and milked/scrubbed for 30 seconds followed by a 30 seconds shower rinse (with gentle manipulation at top of switch to ensure uniform rinsing). Concentrated liquid foam conditioners are applied at 0.033 grams of product per gram of hair with a spatula (foam is dispensed in weigh boat and applied weight recorded) and following the same application procedure.
4. The hair is then dried in a heat box set at 60C for ˜45 minutes or until mostly dry before starting the next treatment cycle or the completion of the treatment cycles.
   For multiple cycle testing, the above procedure is repeated for a set number of times. For instance, for a six cycle test, the above steps 1-4 are repeated six times.

### Deposition Data and Deposition Purity (6 Treatment Cycles):

Deposition Purity may be determined by the ratio of silicone deposited per weight of hair to the total deposition of other ingredients per weight of hair. Silicone is determined by digestion of the hair followed by an analysis with a quantitative elemental technique such as ICP for total silicon and converting to silicone based on the % of silicon in the silicone by weight. The total deposition may be determined by the sum of separate deposition measurements. The separate deposition measurements may include but are not limited to: fatty alcohols, EGDS, quaternized agents and silicone. Typically these measurements involve extracting the hair then separating the ingredients of interest with chromatography and quantifying with an externally calibration based on test solution concentration.
ICP-OES silicone hair digestion method: Hair samples treated with different products are submitted as balls of hair with an average sample size of 0.1 g. These hair samples are then digested using a single reaction chamber microwave digestion system (Milestone Inc., Shelton, Conn.) using a 6:1 HNO₃:H₂O₂ mixture and an aliquot of methyl isobutyl ketone (MIBK) in Teflon digestion vessels. A gentle digestion program with a ramp to 95° C. and a manual vent after cooling below 30° C. is used to facilitate retention of silicon. After dilution to volume, the samples are run against an inorganic silicon calibration curve produced on an Optima 8300 ICP-OES system (Perkin Elmer, Waltham, Mass.) run in the axial mode. The silicon values determined are converted to a concentration of silicone polymer-equivalents deposited on the hair sample using the theoretical silicon concentration of the polymer provided by the manufacturer. An untreated hair sample is analyzed to determine the background concentration of silicon to allow correction if needed. Another untreated hair sample is spiked with a known amount of polymer and analyzed to ensure recovery of the polymer and verify the analysis.

### General Population Hair

TABLE 5
Results of Deposition and Deposition Purity on General
Population Hair after 6 treatment cycles

| | | Amino- | | Amino- | | |
|---|---|---|---|---|---|---|
| | Fatty | silicone | | silicone- | | |
| | Alcohol | Deposition | Alkyl Quat | to-fatty | Oil | Total |
| | Deposition | (ppm) | Deposition | alcohol | Deposition | Deposition |
| Regimen | (ppm) | [% RSD] | (ppm) | ratio | Purity | (ppm) |
| Clarifying Shampoo | 1749 +/− 81 | 1557 +/− 844 | 26 +/− 2 | 0.9 | 47% | 3332 |
| plus Pantene Anti- | | [54%] | | | | |
| breakage Conditioner | | | | | | |
| Clarifying Shampoo | 3.0 +/− 8 | 1341 +/− 449 | 6 +/− 0.4 | 447 | 99% | 1350 |
| plus Ex 1 Conditioner | | [33%] | | | | |
| Clarifying Shampoo | 14 +/− 4 | 1040 +/− 105 | 140 +/− 13 | 74 | 87% | 1194 |
| plus Ex 2 Conditioner | | [10%] | | | | |
| Clarifying Shampoo | 488 +/− 23 | 1334 +/− 82 | 94 +/− 13 | 2.7 | 70% | 1916 |
| plus Ex 3 Conditioner | | [6%] | | | | |
| Clarifying Shampoo | 668 +/− 36 | 1546 +/− 67 | 67 +/− 10 | 2.3 | 68% | 2281 |
| plus Ex 4 Conditioner | | [4%] | | | | |
| Foam Shampoo 1 plus | 8 +/− 3 | 934 +/− 60 | 126 +/− 9 | 117 | 87% | 1068 |
| Ex 2 Conditioner | | [6%] | | | | |
| Foam Shampoo 2 plus | 1 +/− 5 | 996 +/− 58 | 158 +/− 12 | 996 | 86% | 1155 |
| Ex 2 Conditioner | | [4%] | | | | |

### Dyed Hair

TABLE 6
Results of Deposition and Deposition Purity on Dyed Hair after 6 treatment cycles

| | | | | | | | Dyed-to- |
|---|---|---|---|---|---|---|---|
| | | | | | | | General |
| | | | | Amino- | | | Population |
| | Fatty | Amino- | Alkyl | silicone- | | | Hair |
| | Alcohol | silicone | Quat | to-fatty | Oil | Total | Deposition |
| | Deposition | Deposition | Deposition | alcohol | Deposition | Deposition | Ratio |
| Regimen | (ppm) | (ppm) | (ppm) | ratio | Purity | (ppm) | (×100%) |
| Clarifying Shampoo | 1532 +/− 84 | 1057 +/− | 32 +/− 3 | 0.7 | 40% | 2621 | 68% |
| plus Pantene Anti- | | 260 [25%] | | | | | |
| breakage Conditioner | | | | | | | |
| Clarifying Shampoo | 31 +/− 16 | 1464 +/− 78 | 11 +/− 0 | 47 | 97% | 1506 | 109% |
| plus Ex 1 Conditioner | | [5%] | | | | | |
| Clarifying Shampoo | 31 +/− 16 | 1385 +/− | 149 +/− 26 | 45 | 88% | 1565 | 133% |
| plus Ex 2 Conditioner | | 149 [11%] | | | | | |
| Clarifying Shampoo | 444 +/− 12 | 2040 +/− | 115 +/− 25 | 4.6 | 78% | 2599 | 153% |
| plus Ex 3 Conditioner | | 147 [7%] | | | | | |
| Clarifying Shampoo | 636 +/− 41 | 2225 +/− | 84 +/− 13 | 3.5 | 76% | 2945 | 144% |
| plus Ex 4 Conditioner | | 108 [5%] | | | | | |
| Foam Shampoo 1 plus | 7 +/− 17 | 1484 +/− 47 | 133 +/− 15 | 212 | 91% | 1624 | 159% |
| Ex 2 Conditioner | | [3%] | | | | | |
| Foam Shampoo 2 plus | 9 +/− 10 | 1479 +/− | 195 +/− 16 | 164 | 88% | 1683 | 148% |
| Ex 2 Conditioner | | 103 [7%] | | | | | |

### General Population Hair Wet Combing, Dry Combing and Hair Volume Data (6 Treatment Cycles):

Wet combing, dry combing and hair volume was assessed of the hair tresses after the 6 treatment cycles via a sensory panel encompassing 12 individuals:
Wet Combing Test (on the day of the final treatment cycle): After the last treatment cycle, the treated hair tresses were wrapped in aluminum foil and labeled in groups. During the panel, a hair tress from each leg grouping was hung on a metal bar and with each switch being detangled with the wider spacing teeth on a professional comb. The panelists then evaluated the ease of wet combing of the switches using the ‘small end’ of a professional comb (using gloved hand to stabilize switch while combing if needed) and record scores on the provided evaluation form (0-10 scale). After all 5 sets of hair have been combed (2 panelists per hair set), hang carts with hair in CT room (50% RH, 70F).
Dry Combing Test (at least one day after the wet combing test): The dried hair switches from each treatment group were placed in separate metal holders hanging side by side on a metal bar. The panelists evaluated the ease of dry combing of the switches using the ‘small end’ of a professional comb and record scores on the provided evaluation form (0-10 scale; 2 panelists per hair set).

### General Population Hair

TABLE 7
Wet/Dry Combing and Hair Volume on General Population Hair after 6 treatment cycles

| | Regimen weight ratio | | | |
|---|---|---|---|---|
| | of oil to high melting | | | |
| | point fatty | Wet | Dry | Hair |
| Regimen | compounds | Combing | Combing | Volume |
| Clarifying Shampoo Control | — | 2.0 | 2.6 | 8.5 |
| Clarifying Shampoo plus Pantene | 32.5:67.5 | 8.2 | 9.8 | 4.3 |
| Anti-breakage Conditioner | | | | |
| Clarifying Shampoo plus Ex 1 Conditioner | 100:0 | 9.0 | 8.0 | 7.6 |
| Clarifying Shampoo plus Ex 2 Conditioner | 100:0 | 9.4 | 8.2 | 5.6 |
| Clarifying Shampoo plus Ex 3 Conditioner | 80:20 | 9.3 | 8.6 | 4.6 |
| Clarifying Shampoo plus Ex 4 Conditioner | 67:33 | 9.2 | 8.3 | 3.6 |
| Foam Shampoo 1 plus Ex 2 Conditioner | 100:0 | 8.6 | 7.1 | 6.0 |
| Foam Shampoo 2 plus Ex 2 Conditioner | 100:0 | 8.9 | 7.5 | 7.5 |

The above deposition data on undamaged general population hair (close to virgin hair) after 6 treatment cycles demonstrates the regimens involving a foam conditioner of the present invention to provide very good wet combing performance (from 8.2 to 9.4 average scores) and dry combing performance (from 7.1 to 8.6 average scores) versus the liquid control regimen (wet combing of 8.2 and dry combing of 9.8). But, importantly the regimens regimens involving a foam conditioner of the present invention of were able to do this with very good hair volume performance after the end of the treatment cycles (hair volume average scores of 3.6 to 7.6) relative to the liquid regimen control (hair volume of 4.3). One can also see that the hair volume trends with the weight ration of oil to high melting point fatty compounds ratio within the regimen compositions (and with 100:0 ratios providing the best hair volume performance). Without being bound to theory, this is hypothesized to be due to significantly less co-deposits of high melting point fatty compounds.

### Dyed Hair

TABLE 8
Wet/Dry Combing and Hair Volume on Dyed Hair after 6 treatment cycles

| | Regimen weight ratio | | | |
|---|---|---|---|---|
| | of oil to high melting | | | |
| | point fatty | Wet | Dry | Hair |
| Regimen | compounds | Combing | Combing | Volume |
| Clarifying Shampoo Control | — | 1.6 | 1.5 | 5.5 |
| Clarifying Shampoo plus Pantene Anti- | 32.5:67.5 | 8.0 | 9.6 | 4.1 |
| breakage Conditioner | | | | |
| Clarifying Shampoo plus Ex 1 Conditioner | 100:0 | 9.3 | 7.6 | 8.1 |
| Clarifying Shampoo plus Ex 2 Conditioner | 100:0 | 9.8 | 8.5 | 4.3 |
| Clarifying Shampoo plus Ex 3 Conditioner | 80:20 | 9.4 | 8.5 | 5.9 |
| Clarifying Shampoo plus Ex 4 Conditioner | 67:33 | 9.3 | 8.7 | 3.9 |
| Foam Shampoo 1 plus Ex 2 Conditioner | 100:0 | 8.7 | 7.7 | 6.2 |
| Foam Shampoo 2 plus Ex 2 Conditioner | 100:0 | 8.6 | 7.5 | 7.1 |

The above deposition data on dyed hair after 6 treatment cycles demonstrates the regimens involving a foam conditioner of the present invention to provide very good wet combing performance (from 8.6 to 9.3 average scores) and dry combing performance (from 7.5 to 8.5 average scores) comparable to the liquid control regimen (wet combing of 8.0 and dry combing of 9.6). But, importantly the regimens regimens involving a foam conditioner of the present invention of were able to do this without very good hair volume performance after the end of the treatment cycles (hair volume average scores of 3.9 to 8.1) relative to the liquid regimen control (hair volume of 4.1). One can also see that the hair volume trends with the weight ration of oil to high melting point fatty compounds ratio within the regimen compositions (and with 100:0 ratios providing the best hair volume performance). Without being bound to theory, this is hypothesized to be due to significantly less co-deposits of high melting point fatty compounds.

### Wet Feel Results of Liquid vs Foam Shampoo

TABLE 9
Wet Friction of liquid vs foam concentrated cleansing shampoo

| | Ex 21 | Ex 22 | Ex 23 | Ex 31 | Ex 32 | Ex 33 |
|---|---|---|---|---|---|---|
| | Liquid Shampoo | | | Foam Shampoo | | |
| Wet Friction (gf) | 2330 | 2143 | 1745 | 1911 | 1777 | 1190 |
| | No | Low | Guar + | No | Low | Guar + |
| | polymer | MW | PQ6 + | polymer | MW | PQ6 + |
| | | Guar | Silicone | | Guar | Silicone |

### Wet Feel Results of Liquid vs. Foam Hair Conditioner

TABLE 10
Wet Friction of liquid vs foam concentrated hair conditioner

| | Cleaned with liquid | | Cleaned with aerosol foam | |
|---|---|---|---|---|
| | shampoo Ex 21 | | shampoo Ex 31 | |
| Hair Shampoo | Ex51 | Ex53 | Ex61 | Ex63 |
| Hair conditioner | Liquid conditioner | | Aerosol foam conditioner | |
| Wet Friction (gf) | 1906 | 1787 | 1615 | 1560 |
| Silicone:FAOH | 100:0 | 67:33 | 100:0 | 67:33 |
| Si deposition | 100 | 67 | 100 | 67 |
| Purity (%) | | | | |
| | Silicone micro- | Fatty alcohol + | Silicone micro- | Fatty alcohol + |
| | emulsion only | CTAC | emulsion only | CTAC |

### Hair Wet Feel Friction Measurement of Shampoo

A switch of 4 grams general population hair at 8 inches length is used for the measurement. Water temperature is set at 100° F., hardness is 7 grain per gallon, and flow rate is 1.6 liter per minute. For shampoos in liquid form, 0.2 ml of a liquid shampoo is applied on the hair switch in a zigzag pattern uniformly to cover the entire hair length, using a syringe. For shampoo in aerosol foam form, foam is dispensed to a weighing pan through an aluminum can of 53×190 mm size from CCL container. O.2 gram of foam shampoo is applied on the hair switch uniformly to cover the entire hair length via a spatula. The hair switch is then 1^{st} lathered for 30 seconds, rinse with water for 30 seconds, and 2^{nd} lathered for 30 seconds. Water flow rate is then reduced to 0.2 liter per minute. The hair switch is sandwiched with a clamp under 1200 gram of force and pulled through the entire length while the water is running at the low flow rate. The pull time is 30 second. Friction is measured with a Friction analyzer with a load cell of 5kg. Repeat the pull under rinse for total of 21 times. Total 21 Friction values are collected. The hair wet Feel Friction of shampoo reported here is the final rinse friction which is the average friction of the last 7 points.

### Hair Wet Feel Friction Measurement of Conditioner

The shampooed hair switch above is used for the measurement. Water temperature is set at 100° F., hardness is 7 grain per gallon, and flow rate is 0.2 liter per minute. For conditioner in liquid form, 0.13 ml of a liquid conditioner is applied on the hair switch in a zigzag pattern uniformly to cover the entire hair length, using a syringe. For conditioner in aerosol foam form, foam is dispensed to a weighing pan through an aluminum can of 53×190 mm size from CCL container. 0.13 gram of foam conditioner is applied on the hair switch uniformly to cover the entire hair length via a spatula. The hair switch is then milked for 30 seconds. Then, it's sandwiched with a clamp under 1200 gram of force and pulled through the entire length while the water is running at the low flow rate. The pull time is 30 second. Friction is measured with a Friction analyzer with a load cell of 5 kg. Repeat the pull under rinse for total of 24 times. Total 24 Friction values are collected. The hair wet Feel Friction of conditioner reported here is the final rinse friction which is the average friction of the last 7 points

### Cone/Plate Viscosity Measurement

The viscosities of the examples in Table 1-4 are measured by a Cone/Plate Controlled Stress Brookfield Rheometer R/S Plus, by Brookfield. Engineering Laboratories, Stoughton, Mass. The cone used (Spindle C-75-1) has a diameter of 75 mm and 1° angle. The viscosity is determined using a steady state flow experiment at constant shear rate of 2 s⁻¹ and at temperature of 26.5° C. The sample size is 2.5 ml and the total measurement reading time is 3 minutes.

### Shampoo Composition Viscosity Reducing Agents

Now referring to Tables 11 and 12, the base formulations were made by mixing 1.6% perfume, 24% linear sodium undecyl sulfate (CAS# 1072-24-8) active, 6% lauramidopropyl betaine (CAS# 4292-10-8) active and 60.4% deionized water which leaves 8% unfilled for the addition of a viscosity reducing agent (the balance being filled in by distilled water). For all compositions, the surfactant, water and additives including viscosity reducing agents were expected to be in a single phase. The viscosity for formulations that showed hazing or clouding and compositions that appeared macroscopically heterogeneous (e.g. multiple layers) at room temperature were not included in the data (represented by N/A).

The agents from Tables 11 and 12 were added to the base formulation at a percentage of 2%, 4%, 6%, and 8% into the above surfactant solution. The subsequent formulations were vortexed and put into oven at 60° C. overnight to form homogeneous solution. The viscosities of the formulations were measured with calibrated viscometers (Size 200/350/450) from Cannon Instrument Company (2139 High Tech Road, State College, Pa., USA, 16803). Prior to the measurement, the formulations were equilibrated in the viscometer reservoir for 30 min at 40° C. in water bath to ensure a homogeneous temperature was reached in the system.

After the equilibration, the formulations were drawn to reach the starting mark with a rubber suction bulb and the flow time between the starting mark and end mark was recorded for calculation. Each formulation was measured three times to calculate average and standard deviation. Between samples, viscometer was cleaned with water and acetone to rinse off residual. The results appear in Table 11.

Viscosity values in Table 11 and Table 12 were calculated based on the equation:

Viscosity (mm²/s. (cSt))=Time (s)*Constant (mm²/s². (cSt/s))

The time in the above equation is the flow time recorded in the experiment and the constant numbers for each calibrated viscometer were obtained from the manuals.

The partition dispersion coefficient (PDC) was calculated using the following equation:

PDC=logP−0.3001*(δD)²+10.362*δD−93.251

wherein logP is the octanol water partitioning coefficient as computed by the Consensus algorithm implemented in ACD/Percepta version 14.02 by Advanced Chemistry Development, Inc. (ACD/Labs, Toronto, Canada), and wherein δD is the Hansen solubility dispersion parameter in (MPa)^{1/2} computed using Steven Abbott and Hiroshi Yamamoto's “HSPIP—Hansen Solubility Parameters in Practice” program, 4^{th} Edition, version 4.1.07.

TABLE 11

| | | | | | Max # of |
|---|---|---|---|---|---|
| | Lowest | | Octanol/Water | | acyclic |
| | viscosity | | Partition | # of | interconnecting |
| | achieved | | Coefficient | polar | SP3 carbon |
| Agent name | (cSt) | PDC | logP | group | atoms |
| Glycerin | 412 | −5.86 | −1.73 | 3 | 3 |
| Dipropylene Glycol | 53 | −4.49 | −0.62 | 3 | 3 |
| Ethanol | 29 | −4.37 | −0.23 | 1 | 2 |
| Raspberry Ketone | 33 | −2.92 | 1.43 | 2 | 2 |
| Triethyl Citrate | 41 | −2.75 | 1.09 | 4 | 2 |
| 5-Methyl-3-Heptanone Oxime | 246 | −2.54 | 2.00 | 1 | 5 |
| Hydroxycitronellal | 34 | −2.01 | 2.08 | 2 | 9 |
| Camphor Gum | 127 | −1.32 | 2.49 | 1 | 1 |
| 2-Isopropyl-5-methyl-2-hexenal | 332 | −1.25 | 3.20 | 1 | 4 |
| Eucalyptol | 203 | −1.08 | 2.85 | 1 | 1 |
| 1,1-Dimethoxyoctane | 308 | −0.96 | 3.58 | 2 | 7 |
| Isobutyl Hexanoate | 109 | −0.94 | 3.60 | 1 | 5 |
| Dihyro Iso Jasmonate | 119 | −0.92 | 2.89 | 2 | 6 |
| Cyclaprop | 404 | −0.50 | 3.31 | 2 | 2 |
| Orange Flower Ether | 419 | −0.30 | 3.78 | 2 | 3 |
| Iso Butyl Salicylate | 409 | −0.30 | 3.67 | 2 | 4 |
| Butyl Salicylate | 368 | −0.21 | 3.76 | 2 | 4 |
| Citronellyl Acetate | 363 | −0.02 | 4.07 | 1 | 6 |
| 3,7-Dimethyloctyl Acetate | 446 | 0.01 | 4.55 | 1 | 10 |
| Iso Bornyl Propionate | 463 | 0.01 | 3.95 | 1 | 2 |
| Veloutone | 47 | 0.10 | 4.00 | 1 | 5 |
| Isoamyl Salicylate | 193 | 0.13 | 4.02 | 2 | 4 |
| gamma-Terpinene | 32 | 0.20 | 4.10 | 2 | 3 |
| Linalyl Iso Butyrate | 38 | 0.27 | 4.41 | 2 | 4 |
| alpha-Terpinene | 42 | 0.44 | 4.38 | 2 | 3 |
| Limonene | 86 | 0.50 | 4.40 | 2 | 1 |
| Dipentene | 35 | 0.50 | 4.40 | 2 | 1 |
| Geranyl Phenyl Acetate | 34 | 1.65 | 5.47 | 1 | 2 |
| Iso Propyl Myristate | 30 | 3.13 | 7.41 | 1 | 13 |
| Hexadecane | 18 | 4.37 | 8.74 | 0 | 16 |
| 1-Eicosene | N/A | 5.58 | 9.94 | 1 | 18 |

TABLE 12
Kinematic viscosity at 40 C. [cSt]

| | | | | | | | Octanol/Water |
|---|---|---|---|---|---|---|---|
| | | | | | Lowest | | Partition |
| | | | | | viscosity | | Coefficient |
| Name | 2% oil | 4% oil | 6% oil | 8% oil | achieved | PDC | logP |
| Glycerin | 649.2 | 579.7 | 512.3 | 411.5 | 412 | −5.86 | −1.73 |
| Dipropylene Glycol | 321.6 | 141.8 | 109.8 | 52.9 | 53 | −4.49 | −0.62 |
| Ethanol | 205.6 | 111.0 | 50.8 | 29.2 | 29 | −4.37 | −0.23 |
| Raspberry Ketone | 151.9 | 107.4 | 54.0 | 33.0 | 33 | −2.92 | 1.43 |
| Triethyl Citrate | 269.4 | 136.6 | 66.6 | 40.5 | 41 | −2.75 | 1.09 |
| 5-Methyl-3- | 246.3 | N/A | N/A | N/A | 246 | −2.54 | 2.00 |
| Heptanone Oxime | | | | | | | |
| Hydroxycitronellal | 310.8 | 145.4 | 65.9 | 34.1 | 34 | −2.01 | 2.08 |
| Camphor Gum | 365.8 | 127.2 | N/A | N/A | 127 | −1.32 | 2.49 |
| 2-Isopropyl-5-methyl- | 332.3 | N/A | N/A | N/A | 332 | −1.25 | 3.20 |
| 2-hexenal | | | | | | | |
| Eucalyptol | 527.3 | 292.9 | 203.1 | N/A | 203 | −1.08 | 2.85 |
| 1,1-Dimethoxyoctane | 500.7 | 308.1 | N/A | N/A | 308 | −0.96 | 3.58 |
| Isobutyl Hexanoate | 390.0 | 177.9 | 140.9 | 109.4 | 109 | −0.94 | 3.60 |
| Dihyro Iso Jasmonate | 334.9 | 119.1 | N/A | N/A | 119 | −0.92 | 2.89 |
| Cyclaprop | 404.2 | N/A | N/A | N/A | 404 | −0.50 | 3.31 |
| Orange Flower Ether | 552.2 | 418.8 | N/A | N/A | 419 | −0.30 | 3.78 |
| Iso Butyl Salicylate | 408.7 | N/A | N/A | N/A | 409 | −0.30 | 3.67 |
| Butyl Salicylate | 367.9 | N/A | N/A | N/A | 368 | −0.21 | 3.76 |
| Citronellyl Acetate | 362.7 | N/A | N/A | N/A | 363 | −0.02 | 4.07 |
| 3,7-Dimethyloctyl | 445.7 | N/A | N/A | N/A | 446 | 0.01 | 4.55 |
| Acetate | | | | | | | |
| Iso Bornyl Propionate | 463.1 | N/A | N/A | N/A | 463 | 0.01 | 3.95 |
| Veloutone | 260.2 | 129.1 | 56.3 | 47.3 | 47 | 0.10 | 4.00 |
| Isoamyl Salicylate | 460.3 | 193.2 | N/A | N/A | 193 | 0.13 | 4.02 |
| gamma-Terpinene | 238.0 | 65.0 | 31.7 | N/A | 32 | 0.20 | 4.10 |
| Linalyl Iso Butyrate | 223.4 | 73.0 | 37.7 | 39.9 | 38 | 0.27 | 4.41 |
| alpha-Terpinene | 324.4 | 96.0 | 41.5 | 46.7 | 42 | 0.44 | 4.38 |
| Limonene | 366.5 | 166.8 | 97.5 | 86.4 | 86 | 0.50 | 4.40 |
| Dipentene | 307.6 | 74.5 | 35.1 | 42.6 | 35 | 0.50 | 4.40 |
| Geranyl Phenyl | 224.0 | 76.0 | 34.3 | 36.6 | 34 | 1.65 | 5.47 |
| Acetate | | | | | | | |
| Iso Propyl Myristate | 234.2 | 53.7 | 30.4 | 45.0 | 30 | 3.13 | 7.41 |
| Hexadecane | 21.1 | 18.1 | N/A | N/A | 18 | 4.37 | 8.74 |
| 1-Eicosene | N/A | N/A | N/A | N/A | N/A | 5.58 | 9.94 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) A method of treating the hair, the method comprising:
a) applying to the hair a shampoo composition comprising:
i) from about 8% to about 40% of one or more anionic surfactants, by weight of the shampoo composition;
ii) from about 0.5% to about 15% of a co-surfactant selected from the group consisting of amphoteric, non-ionic, zwitterionic, and combinations thereof;
iii) from about 1% to about 10% propellant, by weight of the shampoo composition;
iv) water;
v) perfume;
wherein the shampoo composition comprises less than 0.25% of one or more shampoo high melting point fatty compounds selected from the group consisting of fatty alcohols, fatty acids, fatty esters, and mixtures thereof;
wherein the shampoo is a single phase;
b) applying the dosage of foam to the hair;
c) rinsing the dosage of foam from the hair;
d) applying to the hair a concentrated conditioner composition dispensed from an aerosol foam dispenser as a dosage of foam, wherein the concentrated conditioner composition comprises:
i) from about 4% to about 22% one or more silicones, by weight of the concentrated conditioner composition, wherein the particle size of the one or more silicones is from about 1 nm to about 300 nm; and wherein the one or more silicones are selected from the group consisting of amino morpholino silicones, aminosilicones, quaternary ammonium polyalkylene oxide silicones, and combinations thereof;
ii) less than 4% of one or more conditioner high melting point fatty compounds, by weight of the concentrated conditioner composition; wherein the high melting point fatty compound is selected from the group consisting of fatty alcohols, fatty acids, fatty esters, and mixtures thereof;
iii) from about 1% to about 10% propellant, by weight of the concentrated conditioner composition;
iv) from about 1% to about 6% perfume, by weight of the concentrated conditioner composition; and
v) from about 50% to about 95% water, by weight of the concentrated conditioner composition;
wherein the concentrated conditioner composition has a liquid phase viscosity of from about 1 centipoise to about 10,000 centipoise; and
wherein the foam has a density of from about 0.025 g/cm³ to about 0.30 g/cm³ when dispensed from the aerosol foam dispenser;
e) rinsing the concentrated conditioner composition from the hair;
wherein the method has a deposition purity of from about 40% to about 100%.

2) The method of claim 1, wherein the deposition purity is from about 50% to about 100%.

3) The method of claim 1, wherein the deposition purity is from about 60% to about 100%.

4) The method of claim 1, wherein the co-surfactant is selected from the group consisting of amphoteric, zwitterionic, and combinations thereof.

5) The method of claim 1, wherein the concentrated conditioner composition comprises from about 5% to about 20% of the silicone, by weight of the concentrated conditioner composition.

6) The method of claim 1, wherein the concentrated conditioner composition comprises less than 3% conditioner high melting point fatty compounds, by weight of the concentrated conditioner composition.

7) The method of claim 1, wherein the concentrated conditioner composition comprises 0% conditioner high melting point fatty compounds, by weight of the concentrated conditioner composition.

8) The method of claim 1, wherein the concentrated conditioner composition is substantially free of conditioner high melting point fatty compounds, by weight of the concentrated conditioner composition.

9) The method of claim 1, wherein the concentrated conditioner composition comprises less than 2% conditioner high melting point fatty compounds, by weight of the concentrated conditioner composition.

10) The method of claim 1, wherein the foam has a dosage weight of from about 1 g to about 5 g when dispensed from the aerosol foam dispenser.

11) The method of claim 1, wherein the density of the foam is from about 0.035 g/cm³ to about 0.20 g/cm³.

12) The method of claim 1, wherein the density of the foam is from about 0.05 g/cm³ to about 0.15 g/cm³.

13) The method of claim 1, wherein the shampoo composition has a viscosity of from about 1 centipoise to about 7,500 centipoise.

14) The method of claim 1, wherein the shampoo composition has a viscosity of from about 5 centipoise to about 5,000 centipoise.

15) The method of claim 1, wherein the shampoo composition has a viscosity of from about 10 centipoise to about 2,500 centipoise.

16) The method of claim 1, wherein the reservoir is integrated with the aerosol foam dispenser.

17) The method of claim 1 wherein the propellant in the conditioner composition comprises trans-1,3,3,3-tetrafluoropropene.

18) A method of treating the hair, the method comprising:
a) providing an aerosol dispenser wherein the dispenser comprises a reservoir adapted for holding a shampoo composition wherein the shampoo composition comprises:
i) from about 22% to about 26% sodium undecyl sulfate;
ii) from about from about 4% to about 9% of a zwitterionic co-surfactant selected from the group consisting of lauramidopropyl betaine, cocoamidopropyl betaine, and combinations thereof;
iii) from about 2% to about 5% propellant, by weight of the shampoo composition;
iv) water;
v) perfume;
wherein the shampoo is a single phase;
b) dispensing the shampoo composition from the aerosol container as a dosage of foam;
c) applying the dosage of foam to the hair;
d) rinsing the dosage of foam from the hair;
e) applying to the hair a concentrated conditioner composition dispensed from an aerosol foam dispenser as a dosage of foam, wherein the concentrated conditioner composition comprises:
i) from about 4% to about 8% aminosilicone, by weight of the concentration conditioner composition;
ii) from 0% to less than 5% fatty alcohol, by weight, wherein the fatty alcohol is selected from the group consisting of cetyl alcohol, stearyl alcohol, and combinations thereof;
iii) from about 2% to about 5% propellant, by weight of the shampoo composition;
iv) from about 2% to about 5% perfume, by weight of the concentrated conditioner composition; and
v) from about 50% to about 95% water, by weight of the concentrated conditioner composition;
f) rinsing the concentrated conditioner composition from the hair;
wherein the method has a deposition purity of from about 40% to about 100%.

19) The method of claim 18, wherein the deposition purity is from about 50% to about 100%.

20) The method of claim 18, wherein the deposition purity is from about 60% to about 100%.
